(19) United States
(12) Patent Application Publication
(10) Pub. No.: US 2009/0012052 A1
(43) Pub. Date: Jan. 8, 2009

(54) METHOD FOR TREATING ER+ BREAST CANCER

(75) Inventors: Robert Mark Coopersmith, Norfolk County, MA (US); David William White, Plymouth County, MA (US); Shengfang Jin, Norfolk County, MA (US); Denzyl Fernandes, Santa Rosa, CA (US); Xuena Lin, Acton, MA (US)

Correspondence Address: HARNESS, DICKEY, & PIERCE, P.L.C, 7700 Bonhomme, Suite 400, ST. LOUIS, MO, 63105, US

(73) Assignee: Ore Pharmaceuticals Inc., Gaithersburg, MD (US)

(21) Appl. No.: 12/100,053

(22) Filed: Apr. 9, 2008

Related U.S. Application Data
(63) Continuation-in-part of application No. 11/935,870, filed on Nov. 6, 2007.
(60) Provisional application No. 60/865,094, filed on Nov. 9, 2006.

(51) Int. Cl. A61K 31/4184; A61K 31/135; A61K 31/56; A61P 35/00; A61K 31/41

(52) U.S. Cl. 514/178; 514/648; 514/381; 514/394

(57) ABSTRACT

A method for selecting a female breast cancer patient for AT₁ receptor antagonist therapy comprises (a) determining whether the cancer comprises a tumor that is ER+ and/or PR+; and (b) selecting the patient for AT₁ receptor antagonist therapy only if the cancer is determined to comprise an ER+ and/or PR+ tumor. A method for treating breast cancer in a female patient further comprises (c) administering to the patient, if so selected, an AT₁ receptor antagonist according to a regimen effective to reduce growth, invasiveness and/or metastasis of the tumor.

This application is a continuation-in-part of application Ser. No. 11/935,870, filed on Nov. 6, 2007, which claims the benefit of U.S. provisional patent application Ser. No. 60/865,094, filed on Nov. 9, 2006, the entire disclosure of each of which is incorporated by reference herein.

### FIELD OF THE INVENTION

The present invention relates to pharmacotherapy for breast cancer and to methods of screening patients for such pharmacotherapy.

### BACKGROUND

The United States has the highest reported incidence of breast cancer in the world, followed closely by western European countries including Iceland, Italy, France, Sweden and the United Kingdom. Incidence has historically been lower in eastern Europe, the Middle East and Asia, but some Asian countries such as Japan and Singapore have seen a two-fold increase over the past few decades.

Breast cancer will be diagnosed in about 13% of women in the U.S. in their lifetimes, and more than 3% will die from the disease. Worldwide, breast cancer is now the leading cause of cancer mortality in women, accounting for more than 400,000 deaths per year. In 2002, more than 1.15 million new cases were diagnosed worldwide, with more than 200,000 of these in the U.S. alone.

Cancerous tumors can arise in any tissue of the breast, but most commonly in epithelial tissue of the lobules and ducts. The epithelial cells are separated from the connective tissue surrounding the lobules and ducts by a layer of extracellular material known as the basement membrane. Tumors that are limited by the basement membrane, but may proliferate in the lumen of a lobule or duct, are referred to as lobular carcinoma in situ (LCIS) or ductal carcinoma in situ (DCIS). LCIS is typically not detected by examination or mammography, whereas DCIS tumors often develop central necrosis and calcify, becoming clinically palpable and/or detectable by mammography. DCIS is more likely than LCIS to be malignant and to become invasive.

However, in situ carcinoma is more important as a predictive marker for invasive cancer than as a disease state in its own right. A lobular or ductal carcinoma is considered to be invasive or infiltrative when it is not limited by the basement membrane. About 75% of breast cancers are diagnosed as infiltrative ductal carcinoma. Such cancers have a tendency to metastasize (spread) via the lymphatic system to other tissues and organs, where they form secondary tumors that can be more deadly than the primary tumors in the breast where the cancer originated.

Typical or presumed progression from normal through precancerous to infiltrative ductal carcinoma of the breast can be summarized:
normal→hyperplasia→DCIS→infiltrative ductal carcinoma

Infiltrative carcinoma can be diagnosed as Stage I, II, III and IV. Stage I is defined by an infiltrative tumor up to 2 cm in size, without spread to the lymph nodes. Stage II is defined by a tumor from 2 to 5 cm in size or by spread to the underarm lymph nodes without sticking of the nodes to one another or to surrounding tissue. At Stage III the tumor is over 5 cm in size or there is clumping or sticking of the lymph nodes to surrounding tissue. At stage IV the cancer has spread to tissues outside the breast and underarm lymph nodes.

Breast tumors often, but do not always, have hormone receptors, more particularly estrogen and progesterone receptors, that can be detected in tissue samples obtained by biopsy. A tumor in which estrogen receptors (ER) are identified is said to be estrogen receptor positive (ER+), and one lacking ER is said to be estrogen receptor negative (ER−). Likewise, tumors can be progesterone receptor positive (PR+) or negative (PR−). Tumors that are ER+ and/or PR+ typically show an increase in rate of proliferation in presence of these respective hormones, which occur naturally in the female body and may be supplemented artificially, for example in hormone replacement therapy (HRT). About 70% of all primary human breast cancers are ER+ and the great majority of these are also PR+.

ER+ breast cancer is often treatable with drugs that bind more or less selectively to ER. Such drugs partially or completely prevent estrogen from binding to ER and thereby modulate a cascade of events leading to cell proliferation and tumor growth. Tamoxifen was the first, and is still most widely used, of a class of such drugs known as selective estrogen receptor modulators (SERMs). SERMs are useful not only in palliative treatment of ER+breast cancer but have marked prophylactic utility in healthy subjects at high risk of developing breast cancer, for example subjects having family history of the disease or a previous finding of atypical hyperplasia or in situ carcinoma in a breast tissue biopsy. Other risk factors include advanced age (e.g., 60 years or older), nulliparity and early menarche. For instance, tamoxifen is widely prescribed for women having one or more risk factors and has been found in extensive studies to reduce incidence of invasive breast cancer, for example by almost 50% when administered for 5 years in the Breast Cancer Prevention Trial (P-1) initiated in 1992. See Fisher et al. (1998) J. Natl Cancer Inst. 90(18):1371-1388.

Another SERM, raloxifene, has likewise been found to have prophylactic value in reducing incidence of invasive breast cancer, at least in postmenopausal women. See Cummings et al. (1999) JAMA 281(23):2189-2197.

Unfortunately, SERMs are not universally effective in preventing or treating breast cancer. Aside from lacking useful effect in ER− cancers, it is now well established that even ER+ cancers can be resistant to SERM therapy. About 40% of ER+ breast cancer patients do not respond to anti-hormone therapy. See for example Biswas et al. (1998) Mol. Med. 4(7):454-467.

Such resistance can be de novo or can be acquired, for example in the course of SERM therapy that initially is effective. See for example Dowsett et al. (2005) Endocrine-Related Cancer 12:S113-S117.

Mullick & Chambon (1990) Cancer Res. 50(2):333-338 reported structural and functional properties of ER in two ER+ breast cancer cell lines, LY2 and T47D, which were said to be resistant to SERMs such as tamoxifen. The ER was reported to be functionally indistinguishable from that of the tamoxifen-sensitive cell line MCF-7. It was concluded that the antiestrogen (i.e., SERM) resistance of LY2 and T47D cell lines arises from an estrogen-independent growth effect.

However, more recently Hoffmann et al. (2004) J. Natl Cancer Inst. 96(3):210-218 presented data showing the IC₅₀ for antiproliferative effect of tamoxifen on estrogen-stimulated breast cancer cells to be lower for T47D (11.8×10⁻⁹ M) than for MCF-7 (45.5×10 M) cell lines.

An option now available for treatment of ER+ invasive breast cancer that is SERM-resistant may be the estrogen receptor antagonist fulvestrant (ICI 182,780), which is believed to down-regulate ER expression in ER+ tumors. See for example Robertson et al. (2001) Cancer Res. 61:6739-6746.

Another approach to treatment of estrogen-sensitive breast cancer is to reduce the level of estrogen circulating in the patient and thereby reduce the amount of estrogen available for binding to ER in breast tissue. This can be accomplished, for example, by inhibition of aromatase, an enzyme involved in biosynthesis of estrogen from androgens. Aromatase inhibitors such as anastrozole, exemestane and letrozole are available for treatment of ER+ invasive breast cancer including such cancer that is or has acquired resistance to SERM therapy.

A body of literature now implicates the peptide angiotensin II (Ang II), a major regulator of blood pressure and cardiovascular homeostasis, in regulation of cell proliferation, angiogenesis, inflammation and tissue remodeling, and it has been suggested that Ang II might also play a role in cancer. See for example the review by Deshayes & Nahmias (2005) Trends Endocrinol. Metab. 16(7):293-299.

In an early study by Noguchi et al. (1988) Cancer 62(3):467-473, Ang II reportedly enhanced the anticancer effect of intra-arterial infusion chemotherapy with doxorubicin for breast cancer.

Ang II exerts its bioregulatory effects through interaction with two major types of receptor located on the surface of target cells. These receptors, referred to as Ang II type 1 and type 2 (respectively AT₁ and AT₂) receptors, have been shown to be expressed in a variety of tissues.

A number of AT₁ receptor antagonists and prodrugs thereof, including candesartan, eprosartan, irbesartan, losartan, ohnesartan, telmisartan and valsartan, have been developed for treatment of hypertension, and other useful properties have been identified for these agents. For example, U.S. Pat. No. 6,174,910 to De Gasparo et al. proposes use of such AT₁ receptor antagonists for stimulation of apoptosis and suppression of cell proliferation.

Guerra et al. (1993) Biochem. Biophys. Res. Commun. 193(1):93-99 reported that AT₁ receptors were highly expressed in medroxyprogesterone-induced ductal adenocarcinomas of the mammary gland in mice. Lobular adenocarcinomas reportedly exhibited much lower AT₁ receptor expression.

Inwang et al. (1997) Br. J. Cancer 75(9):1279-1283 reported expression of AT₁ receptors in human breast epithelial cells. In normal tissues and benign tumors, virtually all epithelial cells were reportedly positive for AT₁, but in malignant tumors both positive and negative cells were found.

U.S. Pat. No. 6,465,502 to Bullock et al. (the '502 patent) reports a study of cell lines originating from human breast tissues. The data obtained are stated to demonstrate, inter alia, presence of AT₁ receptors in normal breast tissue, predominantly on ductal myoepithelial cells. However, in breast tissue specimens from 16 patients having invasive breast cancer (14 of which were invasive ductal carcinoma cases), the cancer cells are reportedly found to be negative for the AT₁ receptor in 11 and weakly positive for the AT₁ receptor in 5. However, in all cases the stroma, or connective tissue, is reportedly found to be AT₁ receptor positive. It is concluded therein: “The increased AT₁ receptor expression in mammary ductal myoepithelium [sic] . . . demonstrate that any AT₁ receptor antagonist . . . may be used for treatment of invasive breast carcinoma . . . . Treatment should be considered as adjuvant therapy in combination with surgery, radiotherapy or as palliative therapy with hormonal therapy or other biological response modifiers such as interferons, interleukins, tumor necrosis factors, monoclonal antibodies, etc.” ('502 patent, col. 9, lines 36-50.)

The '502 patent further states: “While clinical examination and mammography suggest breast cancer, it is only the examination of the tissue biopsy which allow to make the diagnosis. The distribution pattern of AT₁ and AT₂ receptors can be used as marker for hyperplasia (location of AT₁ receptors) and for invasive cancer (location of AT₂ receptors) and therefore for the diagnostic of the malignancy of the tumor.” ('502 patent, col. 9, lines 52-58.)

To help elucidate statements in the '502 patent, reference is made herein to a publication (De Paepe et al. (2001) Histochem. Cell Biol. 116:247-254) co-authored by one of the inventors of the '502 patent, and reporting a study of breast tissue specimens including 10 normal controls, 33 cases of hyperplasia, 23 DCIS cases and 25 invasive carcinomas. Epithelial cells were reported to be clearly positive for AT₁ receptor protein in 31 out of 33 hyperplastic tissues and in 18 out of 23 cases of DCIS. In contrast, invasive carcinomas were never shown to express AT₁ receptor protein on the membrane of the tumor cells, but there was always a strong fibrillar signal on the stroma between the invasive tumor cells (De Paepe et al., p. 249). It was further reported that, out of five invasive carcinomas tested by in situ hybridization, three were strongly positive, one weakly positive and one negative for AT₁ mRNA (De Paepe et al., p. 251). It was concluded that “invasive cancer no longer needs the AT₁ expression which then becomes downregulated and can continue to develop without the trophic growth-stimulating influence of angiotensin II” but that “antagonists of AT₁ could be considered as putative inhibitors of the growth of hyperplastic lesions of the breast” (De Paepe et al., p. 253).

De Paepe et al. (2001), supra, additionally studied expression of AT₁ and AT₂ receptors and influence of Ang II on cell proliferation in a cell line derived from normal human mammary epithelium and in two human breast adenocarcinoma cell lines, T47D and SK-BR-3. It was reported that the T47D cell line expressed high levels of the AT₁ receptor and showed significant stimulation of cell growth by Ang II.

Greco et al. (2003) J. Cell. Physiol. 196:370-377 reported that proliferation of cells cultured from invasive ductal carcinomas was stimulated in a dose-dependent manner by Ang II, and that this effect was blocked by the AT₁ receptor antagonist losartan. The cultured cells were reportedly positive for both ER and PR.

Muscella et al. (2003) J. Endocrinol. 173:315-323 reported that in the breast cancer epithelial cell line MCF-7, both AT₁ and AT₂ receptors were expressed, and that Ang II produced a dose-dependent proliferative effect which could be blocked by a specific AT₁ receptor antagonist (DuP 753, i.e., losartan) but not by a specific AT₂ receptor antagonist.

Koh et al. (2005) Carcinogenesis 26:459-464 studied genetic polymorphism in the AT₁ receptor and reported decreased breast cancer risk associated with certain AT₁ receptor genotypes.

Estrogen can regulate AT₁ receptor expression in complex ways in different tissues. For example, Krishnamurthi et al. (1999) Endocrinol. 140(11):5435-5438 reported that AT₁ receptor expression was decreased in the pituitary and adrenal, but increased in the uterus, by estrogen replacement in ovariectomized rats.

Conversely, the concentration of hormone receptors ER and PR in cancer cells can be modulated by an angiotensin converting enzyme (ACE) inhibitor. For example, Small et al (1997) Breast Cancer Res. Treat. 44(3):217-224 studied effects of the ACE inhibitor captopril on ER and PR protein concentration in human mammary ductal carcinoma cell lines T47D (ER+, PR+) and Hs578T (ER−, PR−). Captopril reportedly reduced ER but increased PR, and inhibited [3H]thymidine incorporation (an index of cell proliferation), in T47D cells. No such effects were seen with another ACE inhibitor, lisinopril.

U.S. Patent Application Publication No. 2003/0203834 of Tallant et al. reports that angiotensin (1-7) (Ang(1-7), the N-terminal heptapeptide fragment of Ang II) inhibits tumor growth, and proposes treatment of cancers having an Ang(1-7) receptor, including breast and lung cancers, with an Ang(1-7) receptor agonist.

U.S. Patent Application Publication No. 2004/0127443 of Pershadsingh reports that certain compounds that block the AT₁ receptor are activators of peroxisome proliferator activated receptors (PPARs), specifically PPARγ activators, and proposes that such compounds, which are said to include telmisartan and irbesartan, can be used to treat conditions known to be treatable by drugs that increase PPARγ activity. Diseases known to be responsive to drugs that increase PPARγ activity are said to include, among many others, “proliferative” diseases. It is further proposed therein to use “ARBs” (AT₁ receptor blockers) in prevention and treatment of “diseases mediated through PPAR-dependent regulation of or interaction with related nuclear receptors, including . . . estrogen receptors.” Among a very extensive list of diseases said to be treatable is “[b]reast cancer including estrogen receptor and progesterone receptor positive or negative subtypes, soft tissue tumors.” It is also proposed that the compounds of interest therein can be used for “[p]romoting cell growth and preventing cell death in the aging process.”

U.S. Patent Application Publication No. 2005/0119323 of Kubota et al. proposes inter alia a method for treating or preventing hormone-independent cancer, such as a hormone-independent prostate or breast cancer, comprising administering a compound having an angiotensin II antagonism, or a prodrug or salt thereof. Among examples of such compounds given are losartan, eprosartan, candesartan cilexetil, valsartan, telmisartan, irbesartan, tasosartan and olmesartan medoxomil. “Hormone-independent cancer” is defined therein as referring to “cancer which does not respond to a hormone drug . . . and cancer which has become not to respond to a hormone drug as a result of long term continuation of hormone therapy . . . .”.

U.S. Patent Application Publication No. 2005/0038003 of Gilbert et al. proposes a method of treating and preventing vascular events and circulatory disorders using a combination of dipyridamole, acetylsalicylic acid and an angiotensin II antagonist, for example telmisartan. Indications said to be treatable include “reduced proliferative capacity of the epithelium in lung and breast cancer.”

Pharmacotherapies for cancer, including breast cancer, are not without adverse side effects. This is especially true where a drug is administered in relatively high doses and/or in combination with other drugs, as is often necessary to achieve the desired anticancer effect. For example, AT₁ receptor antagonists, consistent with their use as antihypertensive drugs, have systemic effects on the cardiovascular system and, in sensitive patients, can cause or exacerbate hypotension and impaired renal and/or hepatic function.

A need continues to exist for new pharmacotherapies for breast cancer, especially for some of the more invasive and/or intractable forms of breast cancer such as primary infiltrative ductal carcinoma, more especially for such cancers that are estrogen-sensitive, and even more especially for such estrogen-sensitive cancers that are not responsive or have become resistant to SERM (e.g., tamoxifen) therapy. New modes of treatment and new ways of screening patients to ensure they receive appropriate treatment, would represent an important advance in the art by expanding the range of treatment options now available to the clinician and the breast cancer patient.

### SUMMARY OF THE INVENTION

It has now surprisingly been found that expression of AT₁ receptor mRNA in human breast tissue is dramatically up-regulated in presence of primary infiltrating ductal carcinoma. Even more surprisingly, this up-regulation is seen only in estrogen receptor positive (ER+) and data indicate that this is further augmented in ER+ cancers that are also progesterone receptor positive (PR+). In estrogen receptor negative (ER−) cancers, AT₁ receptor mRNA has been found to be expressed no more highly than in normal breast tissue, and there are even indications that in these ER− cancers there is a lower level of expression of AT₁ receptor mRNA than in normal breast tissue.

Further, it has now been found that, while Ang II induces tumor cell proliferation, and AT₁ receptor antagonists are capable of decreasing Ang II-induced tumor cell proliferation, in an ER+ cell line, neither Ang II nor AT₁ receptor antagonists affect cell proliferation in an ER− cell line.

These results point to ER+/− status being a powerful indicator for responsiveness of a breast cancer to AT₁ receptor antagonist therapy.

Accordingly, there is now provided a method for selecting a female breast cancer patient for AT₁ receptor antagonist therapy, comprising
(a) determining whether the cancer comprises a tumor that is ER+ and/or PR+; and
(b) selecting the patient for AT₁ receptor antagonist therapy only if the cancer is determined to comprise an ER+ and/or PR+tumor.

There is further provided a method for treating breast cancer in a female patient, comprising
(a) determining whether the cancer comprises a tumor that is ER+ and/or PR+;
(b) selecting the patient for AT₁ receptor antagonist therapy only if the cancer is determined to comprise an ER+ and/or PR+ tumor; and
(c) administering to the patient, if so selected, an AT₁ receptor antagonist according to a regimen effective to reduce growth, invasiveness and/or metastasis of the tumor.

Such a regimen can, for example, comprise a daily dose of about 0.01 to about 100 mg/kg of the AT₁ receptor antagonist.

It is contemplated that AT₁ receptor antagonists can be effective in decreasing Ang II-induced cell proliferation in an ER+ tumor regardless of its responsiveness to SERMs such as tamoxifen. This opens up a new option for treatment of ER+ breast cancers that remain estrogen sensitive but are or have become resistant to tamoxifen or other SERMs, for example through long-term preventive administration, and are thus especially challenging.

Accordingly, there is still further provided a method for treating a breast tumor in a female patient having SERM-resistant ER+ breast cancer, comprising administering to the patient an AT₁ receptor antagonist according to a regimen effective to reduce growth, invasiveness and/or metastasis of the tumor.

There is still further provided a method for treating a breast tumor in a female patient, comprising administering to the patient an AT₁ receptor antagonist and a second agent that comprises an aromatase inhibitor or an estrogen receptor modulator or antagonist, in amounts effective in combination to reduce growth, invasiveness and/or metastasis of the breast tumor. The method is particularly appropriate for treating an ER+ breast tumor. Where the breast tumor is ER+ but SERM-resistant, the second agent should advantageously comprise an aromatase inhibitor or an estrogen receptor antagonist.

There is still further provided a therapeutic combination comprising an AT₁ receptor antagonist and a second agent that comprises an aromatase inhibitor or an estrogen receptor modulator or antagonist, in amounts effective in combination to reduce growth, invasiveness and/or metastasis of a breast tumor. Such a combination can be particularly useful for treating an ER+ breast tumor. Again, where the breast tumor is ER+ but SERM-resistant, the second agent should advantageously comprise an aromatase inhibitor or an estrogen receptor antagonist.

There are still further provided kits comprising therapeutic combinations as described above.

Still further provided are various screening or diagnostic methods, including:
a method for screening a patient population for AT₁ receptor antagonist therapy for breast cancer; this method comprises determining, in a breast tissue sample from each of a plurality of patients, whether a tumor that is ER+ and/or PR+ is present; wherein a patient is selected for the therapy only if such a tumor is found to be present; and
a method for identifying a breast having a primary invasive ductal carcinoma and overexpressing an AT₁ receptor by comparison with a normal breast; this method comprises determining whether the carcinoma comprises an ER+ tumor, wherein presence of an ER+ tumor is indicative of AT₁ receptor overexpression in the breast.

Other embodiments, including particular aspects of the embodiments summarized above, will be evident from the detailed description that follows.

### BRIEF DESCRIPTION OF THE DRAWINGS

FIG. 1 presents results of a study, as described in Example 2, comparing an ER+ cell line (T47D) and an ER− cell line (HCC1143) with respect to effect on cell proliferation of Ang II.

FIG. 2 presents results of a study, as described in Example 3, comparing an ER+ cell line (T47D) and an ER− cell line (HCC1143) with respect to effect of an AT₁ receptor antagonist (telmisartan) on Ang II-induced cell proliferation.

FIG. 3 presents results of a study, as described in Example 4, showing effect of an AT₁ receptor antagonist (candesartan) on Ang II-induced cell proliferation in an ER+ cell line (T47D).

FIG. 4 presents results of a study, as described in Example 5, showing effect of an AT₁ receptor antagonist (irbesartan) on Ang II-induced cell proliferation in an ER+ cell line (T47D).

FIG. 5 presents results of a study, as described in Example 13, showing effects of an aromatase inhibitor (formestane), an AT₁ receptor antagonist (irbesartan), and a combination of both on Ang II-induced cell proliferation in an ER+ cell line (T47D).

FIG. 6 presents results of a study, as described in Example 14, showing effects of a SERM (tamoxifen), an AT₁ receptor antagonist (irbesartan), and a combination of both on Ang II-induced cell proliferation in an ER+ cell line (T47D).

FIGS. 7, 8 and 9 present results of an in vivo study, as described in Example 15, showing effects of the AT₁ receptor antagonists candesartan cilexetil (FIG. 7) and irbesartan (FIG. 8) by comparison with the positive control tamoxifen (FIG. 9) on tumor growth in ER+ cell line (T47D) xenografts in mice.

### DETAILED DESCRIPTION

In one embodiment, the present invention provides a method for selecting a female breast cancer patient for AT₁ receptor antagonist therapy. The method of this embodiment comprises (a) determining whether the cancer comprises a tumor that is ER+ and/or PR+; and (b) selecting the patient for AT₁ receptor antagonist therapy only if the cancer is determined to comprise an ER+ and/or PR+tumor.

Step (a) according to this method is referred to herein as the “testing step” and step (b) as the “selection step”. The method is particularly useful where the patient presents with primary infiltrating ductal carcinoma of the breast.

In the testing step, determination of presence of an ER+ and/or PR+tumor can be made in situ, but typically a tissue sample is extracted from the affected breast, for example by biopsy or in the course of surgery, and determination of presence of an ER+ and/or PR+tumor is made in the tissue sample by obtaining a positive result in an assay.

Any assay known in the art for detection of estrogen and/or progesterone receptors can be used. Assay methods include, without limitation, ligand binding assays, immunohistochemical assays (including immunocytochemical assays) and combinations thereof. Reference may be made, for example, to the publications individually cited below and incorporated herein by reference.

Graham et al. (1999) Am. J. Vet. Res. 60:627-630.

Heubner et al. (1986) Cancer Res. 46(8 suppl.):4291s-4295s.

Harvey et al. (1999) J. Clin. Oncol. 17:1474-1481.

In a particular embodiment of the present method, the testing step involves determination of ER+ or ER− status of the cancer, wherein determination of PR+ or PR− status is optional.

It is a feature of the present method that the outcome of the testing step enables a decision to be made, with a high degree of confidence, as to whether the patient will benefit from AT₁ receptor antagonist therapy. It has not heretofore been recognized that beneficial responsiveness of tumor growth, particularly in primary infiltrative ductal carcinoma of the breast, to treatment with an AT₁ receptor antagonist is highly dependent on ER+ and/or PR+status, particularly so on ER+ status, of the tumor. The unexpected discovery in primary infiltrative ductal carcinoma of a close correlation between ER+ status and AT₁ receptor expression (see Example 1 below), together with the finding that only an ER+ cell line (but not an ER− cell line) exhibits Ang II-induced cell proliferation that is inhibited by AT₁ receptor antagonists (see Examples 2-5 below), provides a basis for patient stratification, wherein only patients that have ER+ and/or PR+, more particularly ER+, cancer are selected for AT₁ receptor antagonist therapy.

This represents a significant advance in the art, in a number of ways. For example, AT₁ receptor antagonist therapy, optionally in combination with other intervention as more fully described hereinbelow, can now be targeted to a patient population having a higher probability of successful outcome than without the present testing step. Equally important, a patient population having low probability of successful outcome (e.g., an ER− patient population) can be spared the possibility of adverse side effects associated with AT₁ receptor antagonist therapy and can be directed more efficiently to alternative treatments that are more likely to bring benefit.

Thus, according to the method of the present embodiment, the selection step comprises selecting the patient for AT₁ receptor antagonist therapy only if the cancer is determined to comprise an ER+ and/or PR+tumor. In a particular embodiment, the patient is selected for AT₁ receptor antagonist therapy only if the cancer is determined to comprise an ER+ tumor.

According to the present embodiment, if no ER+ and/or PR+tumor is identified, the patient is selected not to receive AT₁ receptor antagonist therapy. Such a patient may receive no treatment, or more likely an alternative treatment that does not include AT₁ receptor antagonist therapy. Choice of alternative treatment will be made by the clinician in consultation with the patient, based on factors known in the art and not expanded on herein, but which can include, for example, one or more of surgery, radiation therapy and chemotherapy. As the cancer in this case is typically ER−, anti-estrogen treatment will usually not be indicated, although in a recent publication it has been suggested, in view of apparent nongenomic estrogen signaling in ER− breast cancer (possibly involving AT₁ receptors), that aromatase inhibitors may be beneficial in treating ER− as well as ER+ breast tumors. See Lim et al. (2006) Breast Cancer Res. 8(3):R33 (e-publication), incorporated by reference herein but not admitted to be prior art to the present invention.

Optionally, the present method further comprises, if an ER+ tumor is identified, determining whether the tumor is resistant or responsive to treatment with a SERM such as tamoxifen, raloxifene or toremifene. This optional determination step can involve review of patient history; for example, whether a SERM has previously and with incomplete success been administered to the patient (including prophylactic administration). Alternatively or in addition, tumor cells from a tissue sample extracted from the patient can be tested in any suitable in vitro or in vivo assay for SERM resistance. For example, an enzyme immunoassay distinguishing cancers that are tamoxifen-sensitive from cancers having acquired tamoxifen resistance is described by Naundorf et al (2000) Breast Cancer Res. Treat. 60(1):81-92.

In one scenario, an ER+ patient can be selected for AT₁ receptor antagonist therapy whether or not the cancer is determined to be SERM-resistant. However, even in this scenario, the options for combination therapy are likely to be different for a SERM-resistant versus SERM-responsive tumor. For example, a regimen for SERM-responsive cancer can include administration of any one or more anti-estrogen drugs, including SERMs, in combination with the AT₁ receptor antagonist therapy; whereas a regimen for SERM-resistant cancer can include administration of an estrogen antagonist (e.g., fulvestrant) or an aromatase inhibitor (e.g., aminoglutethimide, anastrozole, exemestane, fadrozole, formestane, letrozole or vorozole) in combination with the AT₁ receptor antagonist therapy.

Notwithstanding the above, it is not ruled out a SERM can advantageously be combined with an AT₁ receptor antagonist in treatment of a tumor regardless of its responsiveness to the SERM alone.

As illustrated in Example 14, it has been found that cell proliferation in an ER+ cell line (T47D) in presence of Ang II is reduced by the SERM tamoxifen and by the AT₁ receptor antagonist irbesartan, and that the combination of both agents provides an increased antiproliferative effect by comparison with either agent alone.

Further, as illustrated in Example 13, it has been found that cell proliferation in an ER+ cell line (T47D) in presence of Ang II is reduced by the aromatase inhibitor formestane and by the AT₁ receptor antagonist irbesartan, and that the combination of both agents provides an increased antiproliferative effect by comparison with either agent alone.

In another scenario, an ER+ patient can be selected for AT₁ receptor antagonist therapy only if the cancer is determined to comprise a SERM-resistant ER+ tumor. A rationale for this approach is that where the cancer is determined to be SERM-responsive, there is a relatively high probability of successful treatment with a SERM such as tamoxifen, raloxifene or toremifene, and the incremental benefit of AT₁ receptor antagonist administration may therefore be lower. However, as illustrated in Example 14, even where a strong antiproliferative response is seen to treatment with tamoxifen, the addition of an AT₁ receptor antagonist (in this case irbesartan) can further increase that response.

In yet another scenario, the decision as to inclusion of an AT₁ receptor antagonist in a regimen for SERM-responsive breast cancer depends in part of the degree of invasiveness or stage of the cancer. For example, early-stage SERM-responsive cancer may be adequately treated by a SERM alone, while for more advanced cancer (e.g., stage II or III primary infiltrative ductal carcinoma) there may be significant benefit in combination therapy with a SERM and an AT₁ receptor antagonist.

Unless the context demands otherwise, the term “treat,” “treating” or “treatment” herein includes preventive or prophylactic use of an agent, for example an AT₁ receptor antagonist, in a subject at risk of, or having a prognosis including, breast cancer, as well as use of such an agent in a subject already experiencing breast cancer, as a therapy to alleviate, relieve, reduce intensity of or eliminate one or more symptoms of the disease or an underlying cause thereof. Thus treatment includes (a) preventing a condition or disease from occurring in a subject that may be predisposed to the condition or disease but in whom the condition or disease has not yet been diagnosed; (b) inhibiting the condition or disease, including retarding or arresting its development; and/or (c) relieving, alleviating or ameliorating the condition or disease, or primary or secondary signs and symptoms thereof, including promoting, inducing or maintaining remission of the disease.

In one embodiment, the present invention provides a method for treating breast cancer in a female patient, comprising (a) determining whether the cancer comprises a tumor that is ER+ and/or PR+; (b) selecting the patient for AT₁ receptor antagonist therapy only if the cancer is determined to comprise an ER+ and/or PR+tumor; and (c) administering to the patient an AT₁ receptor antagonist according to a regimen effective to reduce growth, invasiveness and/or metastasis of the tumor. Steps (a) and (b) will be recognized as the same testing and selection steps, respectively, as in the method of the embodiment described above. The same options, variants and specific modalities mentioned above for steps (a) and (b) apply equally to the method of the present embodiment, which further comprises step (c), referred to herein as the “treatment step”. It will be understood that in the method of this embodiment, the term “treatment” does not extend to purely preventive or prophylactic use, as it is required for the treatment step that the patient have a tumor.

Reference herein to testing, selection or treatment of a “primary” cancer or tumor will be understood not to be limited to situations where metastasis has not occurred. A “primary” tumor is thus a tumor at the site of origin of the cancer, regardless of whether secondary tumors occur in other tissues or organs.

An Ang II receptor antagonist (in some literature referred to as an AIIR antagonist) is any compound that binds or otherwise interacts with Ang II receptors to partially or completely block effects of Ang II. As there are at least two types of Ang II receptor, namely AT₁ and AT₂ receptors, an Ang II receptor antagonist can bind or interact with either one or both of these receptor types. Of particular interest herein are AT₁ receptor antagonists (or AT₁R antagonists), which are compounds that bind or interact preferentially or exclusively with the AT₁ receptor to partially or completely block effects of Ang II.

A very large number of Ang II receptor antagonists have been described in the art. For example, a useful illustrative list of such compounds, with reference to sources describing their preparation, may be found in U.S. Patent Application Publication No. 2004/0102423 of MacLaughlan & Schuh, at Table II thereof, which is incorporated herein by reference in its entirety. Any of the compounds listed therein that exhibit AT₁ receptor antagonism, or any pharmaceutically acceptable salts, prodrugs or active metabolites of such compounds, can be used in methods, therapeutic combinations, pharmaceutical compositions and kits of the present invention.

Ang II receptor antagonists useful herein are also described and characterized, with methods of preparation, in the patents and publications individually cited below and incorporated herein by reference.
U.S. Pat. No. 5,015,651 to Carini et al.
U.S. Pat. No. 5,043,349 to Carini et al.
U.S. Pat. No. 5,087,634 to Reitz & Manning.
U.S. Pat. No. 5,098,920 to Reitz.
U.S. Pat. No. 5,100,897 to Allen et al.
U.S. Pat. No. 5,104,891 to Bovy et al.
U.S. Pat. No. 5,124,335 to Patchett et al.
U.S. Pat. No. 5,126,342 to Chakravarty et al.
U.S. Pat. No. 5,128,327 to Chakravarty et al.
U.S. Pat. No. 5,137,902 to Carini.
U.S. Pat. No. 5,138,069 to Carini et al.
U.S. Pat. No. 5,140,036 to Reitz.
U.S. Pat. No. 5,151,435 to Bagley et al.
U.S. Pat. No. 5,155,117 to Reitz.
U.S. Pat. No. 5,157,040 to Greenlee et al.
U.S. Pat. No. 5,162,325 to Chakravarty et al.
U.S. Pat. No. 5,162,326 to Naka & Nishikawa.
U.S. Pat. No. 5,162,340 to Chakravarty et al.
U.S. Pat. No. 5,164,403 to Reitz & Manning.
U.S. Pat. No. 5,175,164 to Bagley et al.
U.S. Pat. No. 5,175,180 to Bovy et al.
U.S. Pat. No. 5,177,074 to Allen et al.
U.S. Pat. No. 5,177,095 to Greenlee et al.
U.S. Pat. No. 5,177,096 to Keenan & Weinstock.
U.S. Pat. No. 5,183,810 to Greenlee et al.
U.S. Pat. No. 5,183,899 to Naka & Nishikawa.
U.S. Pat. No. 5,185,351 to Finkelstein et al.
U.S. Pat. No. 5,187,159 to Greenlee & Rivero.
U.S. Pat. No. 5,187,179 to Ashton et al.
U.S. Pat. No. 5,187,271 to Bovy et al.
U.S. Pat. No. 5,196,444 to Naka et al.
U.S. Pat. No. 5,198,438 to Allen et al.
U.S. Pat. No. 5,196,537 to Reitz.
U.S. Pat. No. 5,202,322 to Allen & Olson.
U.S. Pat. No. 5,202,328 to de Laszlo et al.
U.S. Pat. No. 5,210,204 to Connor & Kostlan.
U.S. Pat. No. 5,210,211 to Hodges & Topliss.
U.S. Pat. No. 5,219,856 to Olson.
U.S. Pat. No. 5,223,501 to Chakravarty et al.
U.S. Pat. No. 5,225,428 to Kramer et al.
U.S. Pat. No. 5,236,928 to Chakravarty et al.
U.S. Pat. No. 5,238,942 to Chakravarty et al.
U.S. Pat. No. 5,240,928 to Allen et al.
U.S. Pat. No. 5,240,938 to Greenlee et al.
U.S. Pat. No. 5,242,939 to Sircar.
U.S. Pat. No. 5,246,943 to Blankley et al.
U.S. Pat. No. 5,248,689 to Girard et al.
U.S. Pat. No. 5,250,521 to Allen et al.
U.S. Pat. No. 5,250,548 to Winn et al.
U.S. Pat. No. 5,250,554 to Naka & Nishikawa.
U.S. Pat. No. 5,254,543 to Hanko et al.
U.S. Pat. No. 5,254,546 to Ardecky et al.
U.S. Pat. No. 5,256,667 to Allen & Olson.
U.S. Pat. No. 5,260,285 to Allen et al.
U.S. Pat. No. 5,262,412 to Ashton et al.
U.S. Pat. No. 5,264,447 to Ohtawa
U.S. Pat. No. 5,266,583 to Ohtawa.
U.S. Pat. No. 5,270,317 to Bernhart et al.
U.S. Pat. No. 5,270,322 to Ries et al.
U.S. Pat. No. 5,276,054 to Diez et al.
U.S. Pat. No. 5,294,633 to Kramer et al.
U.S. Pat. No. 5,318,980 to Hanko et al.
U.S. Pat. No. 5,326,776 to Winn et al.
U.S. Pat. No. 5,330,987 to Allen et al.
U.S. Pat. No. 5,332,744 to Chakravarty et al.
U.S. Pat. No. 5,332,831 to Dowle & Judd.
U.S. Pat. No. 5,352,687 to Müller et al.
U.S. Pat. No. 5,354,749 to Dressel et al.
U.S. Pat. No. 5,356,911 to Müller-Gliemann et al.
U.S. Pat. No. 5,364,942 to Hanko et al.
U.S. Pat. No. 5,374,646 to Ross et al.
U.S. Pat. No. 5,376,671 to Müller-Gliemann et al.
U.S. Pat. No. 5,389,641 to Naka & Inada.
U.S. Pat. No. 5,399,566 to Katano et al.
U.S. Pat. No. 5,399,578 to Bühlmayer et al.
U.S. Pat. No. 5,407,942 to Dressel et al.
U.S. Pat. No. 5,409,936 to Honma et al.
U.S. Pat. No. 5,411,980 to Ashton et al.
U.S. Pat. No. 5,412,097 to Chakravarty et al.
U.S. Pat. No. 5,414,008 to Müller et al.
U.S. Pat. No. 5,436,260 to Hodges & Klutchko.
U.S. Pat. No. 5,444,071 to Roberts et al.
U.S. Pat. No. 5,447,949 to Girard et al.
U.S. Pat. No. 5,459,147 to Hauel et al.
U.S. Pat. No. 5,463,073 to Naka & Inada.
U.S. Pat. No. 5,508,299 to Müller et al.
U.S. Pat. No. 5,512,681 to Boswell et al.
U.S. Pat. No. 5,519,138 to Ries et al.
U.S. Pat. No. 5,530,017 to Girard et al.
U.S. Pat. No. 5,538,987 to Salimbeni et al.
U.S. Pat. No. 5,554,625 to Rivero et al.
U.S. Pat. No. 5,565,464 to Salimbeni et al.
U.S. Pat. No. 5,565,480 to Gleason et al.
U.S. Pat. No. 5,583,141 to Naka & Inada.
U.S. Pat. No. 5,591,762 to Hauel et al.
U.S. Pat. No. 5,616,599 to Yanagisawa et al.
U.S. Pat. No. 5,627,191 to Birch et al.
U.S. Pat. No. 5,654,322 to Hirata et al.
U.S. Pat. No. 5,703,110 to Naka et al.
U.S. Pat. No. 5,728,842 to Hill et al.
U.S. Pat. No. 5,864,043 to Narr et al.
U.S. Pat. No. 6,004,989 to Naka et al.
International Patent Publication No. WO 91/15479.
International Patent Publication No. WO 91/18888.
International Patent Publication No. WO 91/19715.
International Patent Publication No. WO 92/09278.
International Patent Publication No. WO 92/20662.
International Patent Publication No. WO 93/18035.
International Patent Publication No. WO 93/23399.
European Patent Application Publication No. EP 0 412 594.
European Patent Application Publication No. EP 0 430 300.
European Patent Application Publication No. EP 0 434 038.
European Patent Application Publication No. EP 0 443 568.
European Patent Application Publication No. EP 0 470 543.
European Patent Application Publication No. EP 0 502 314.
European Patent Application Publication No. EP 0 537 937.
European Patent Application Publication No. EP 0 539 713.
European Patent Application Publication No. EP 0 556 789.
European Patent Application Publication No. EP 0 563 705.

U.K. Patent Application Publication No. GB 2 264 710.

More particularly, the AT₁ receptor antagonists identified below, including their pharmaceutically acceptable salts, prodrugs and active metabolites, are useful herein.

Candesartan (2-ethoxy-1-[4-[2-(1H-tetrazol-5-yl)phenyl]benzyl]-7-benzimidazole-carboxylic acid) is described and a process for its preparation provided in above-cited U.S. Pat. No. 5,196,444. The 1-[[(cyclohexyloxy)carbonyl]oxy]ethyl ester, known as candesartan cilexetil, is a prodrug available for example as Atacand®.

Eprosartan ((E)-3-[2-butyl-1-[(4-carboxyphenyl)methyl]imidazol-5-yl]-2-(2-thienyl-methyl)-2-propenoic acid) is described and a process for its preparation provided in above-cited U.S. Pat. No. 5,185,351. An illustrative salt is eprosartan mesylate, available for example as Teveten®.

Irbesartan (2-n-butyl-4-spirocyclopentane-1-[(2′-(tetrazol-5-yl)bipheny-4-yl)methyl]-2-imidazolin-5-one) is described and a process for its preparation provided in above-cited U.S. Pat. No. 5,270,317. Irbesartan is available for example as Avapro®.

Losartan (2-n-butyl-4-chloro-5-hydroxymethyl-1-[[2′-(1H-tetrazol-5-yl)biphenyl-4-yl]methyl]imidazole) is described and a process for its preparation provided in above-cited U.S. Pat. No. 5,138,069. An illustrative salt is losartan monopotassium, available for example as Cozaar®.

A metabolite of losartan, EXP-3174 (2-n-butyl-4-chloro-1-[2′-(1H-tetrazol-5-yl)biphenyl-4-yl]methyl-1H-imidazole-5-carboxylic acid), is also active as an AT₁ receptor antagonist. See Lynch et al. (1999) J. Am. Coll. Cardiol. 34(3):876-884.

Olmesartan (4-(1-hydroxy-1-methylethyl)-2-propyl-1-[[2′-(1H-tetrazol-5-yl)-1,1′-biphenyl-4-yl]methyl]-1H-imidazole-5-carboxylic acid) is described and a process for its preparation provided in above-cited U.S. Pat. No. 5,616,599. The (5-methyl-2-oxo-1,3-dioxol-4-yl)methyl ester, known as olmesartan medoxomil, is a prodrug available for example as Benicar®.

Saprisartan (1-[[3-bromo-2-[2-[[(trifluoromethyl)sulfonyl]amino]phenyl]-5-benzo-furanyl]methyl]-4-cyclopropyl-2-ethyl-1H-imidazole-5-carboxamide) is described and a process for its preparation provided in above-cited U.S. Pat. No. 5,332,831.

Tasosartan (5,8-dihydro-2,4-dimethyl-8-(p-(o-1H-tetrazol-5-ylphenyl)benzyl) pyrido(2,3-d)pyrimidin-7(6H)-one), also known as ANA-756, is described and a process for its preparation provided by Ellingboe et al. (1994) J. Med. Chem. 37:542-550.

A metabolite of tasosartan known as enoltasosartan has been identified. See Maillard et al. (2000) J. Pharmacol. Exp. Ther. 295(2):649-654.

Telmisartan (4′-[[4-methyl-6-(1-methyl-2-benzimidazolyl)-2-propyl-1-benzimidazol-yl]methyl]-2-biphenylcarboxylic acid) is described and a process for its preparation provided in above-cited U.S. Pat. No. 5,591,762. Telmisartan is available for example as Micardis®.

Valsartan ((S)—N-(1-carboxy-2-methylprop-1-yl)-N-[2′-(1H-tetrazol-5-yl)biphenyl-4-ylmethyl]amine) is described and a process for its preparation provided in above-cited U.S. Pat. No. 5,399,578. Valsartan is available for example as Diovan®.

Zolarsartan ((1-[[3-bromo-2-[2-(1H-tetrazol-5-yl)-phenyl]-5-benzofuranyl]methyl]-2-butyl-4-chloro-1H-imidazole-5-carboxylic acid) is described and a process for its preparation provided in above-cited U.S. Pat. No. 5,374,646.

KRH-594 ((Z)-2-[[5-ethyl-3-[2′-(1H-tetrazol-5-yl)biphenyl-4-yl]methyl-1,3,4-thiadiazolin-2-ylidene]aminocarbonyl]-1-cyclopentenecarboxylic acid) is described and a process for its preparation provided in above-cited U.S. Pat. No. 5,654,322. KRH-594 can be used for example as its dipotassium salt.

ME-3221 (3-methoxy-2,6-dimethyl-4-[[2′-(1H-tetrazol-5-yl)-1,1′-biphenyl-4-yl]-methoxy]pyridine) is described and a process for its preparation provided in above-cited U.S. Pat. No. 5,399,566.

A metabolite of ME-3221, 3-hydroxy-2,6-dimethyl-4-[[2′-(1H-tetrazol-5-yl)-1,1′-biphenyl-4-yl]methoxy]pyridine, is also active as an AT₁ receptor antagonist. See Nagura et al. (1995) Eur. J. Pharmacol. 274(1-3):201-211.

SC-52458 (5-[(3,5-dibutyl-1H-1,2,4-triazol-1-yl)methyl]-2-[2-(1H-tetrazol-5-yl-phenyl)]pyridine), sometimes known as forasartan, is described and a process for its preparation provided in above-cited U.S. Pat. No. 5,196,537.

TA-606 ((3-pentyloxy)carbonyloxymethyl-5-acetyl-2-n-propyl-3-[2′-(1H-tetrazol-5-yl)biphenyl-4-yl]methyl-4,5,6,7-tetrahydroimidazo[4,5-c]pyridine-4-carboxylic acid) is stated by Hashimoto et al. (1998) J. Cardiovasc. Pharmacol. 31(4):568-575 to be a prodrug exhibiting Ang II receptor antagonist activity. TA-606 can be used for example as its hydrochloride salt.

A related Ang II antagonist, 5-acetyl-2-n-propyl-3-[2′-(1H-tetrazol-5-yl)biphenyl-4-yl]methyl-4,5,6,7-tetrahydroimidazo[4,5-c]pyridine-4-carboxylic acid, is described and a process for its preparation provided in above-cited U.S. Pat. No. 5,409,936.

ZD-8731 (2-ethyl-4-[(2′-(1H-tetrazol-5-yl)biphenyl-4-yl)methoxy]quinoline) is described and a process for its preparation provided in above-cited U.S. Pat. No. 5,444,071.

Other compounds useful as Ang II antagonists herein include, without limitation, A-81282, A-81988, BMS-184,698, CGP-49870, CI-996, CL-329,167, CP-161418, D-8731, DMP-581, DMP-811, DuP-532, E-4177, EMD-66397, EXP-408, EXP-970, EXP-3892, EXP-6803, EXP-7711, GA-0050, GR-138,950, HN-65,021, KT-3671, KW-3433, L-158,809, L-158,978, L-159,282, L-159,686, L-159,689, L-159,874, L-161,177, L-161,816, L-162,154, L-162,234, L-162,441, L-163,007, L-163,017, LF-7-0156, LR-B-081, LY-285,434, MK-996, PD-123,177, PD-123,319, PD-150,304, RWJ-38970, RWJ-46458, SC-48742, SC-50560, SC-51316, SC-51895, SL-910,102, TAK-536, U-96,849, UP-269-6, UP-27,522, UR-7198, WAY-126,227, WK-1492, XH-148, XR-510, YM-358 and YM-31,472, each as identified for example in U.S. Pat. No. 6,218,414 to Nisato incorporated by reference herein, including pharmaceutically acceptable salts, prodrugs and active metabolites of such compounds.

The AT₁ receptor antagonist administered illustratively has a chemical structure that includes a benzylamine moiety

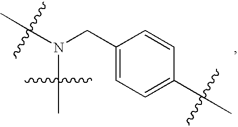

for example such a moiety wherein the nitrogen atom is part of a further substituted imidazole, benzimidazole or imidazolinone ring system. In some examples, the benzylamine moiety can be represented as follows:

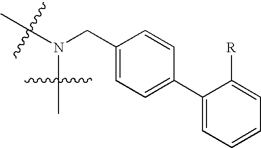

where R can be, without limitation, a partially saturated (e.g., 1H—) 1,2,3,4-tetrazolyl group or a carboxylic acid group.

In one embodiment, the AT₁ receptor antagonist administered comprises at least one compound selected from the group consisting of candesartan, eprosartan, irbesartan, losartan, olmesartan, saprisartan, tasosartan, telmisartan, valsartan, zolarsartan and pharmaceutically acceptable salts, prodrugs and active metabolites thereof.

As reported in above-cited U.S. Patent Application Publication No. 2004/0127443 of Pershadsingh, certain AT₁ receptor antagonists also exhibit PPARγ agonist activity. Such dual activity is not a requirement of the present invention. Thus in some embodiments the AT₁ receptor antagonist administered exhibits PPARγ agonist activity, and in other embodiments the AT₁ receptor antagonist administered exhibits no substantial PPARγ agonist activity, in the target tumor.

Certain compounds useful according to the present invention have acid and/or base moieties that, under suitable conditions, can form salts with suitable acids. Internal salts can also be formed. The compound can be used in its free acid/base form or in the form of an internal salt, an acid addition salt or a salt with a base.

Acid addition salts can illustratively be formed with inorganic acids such as mineral acids, for example sulfuric acid, phosphoric acids or hydrohalic (e.g., hydrochloric or hydrobromic) acids; with organic carboxylic acids such as (a) C₁₋₄ alkanecarboxylic acids which may be unsubstituted or substituted (e.g., halosubstituted), for example acetic acid, (b) saturated or unsaturated dicarboxylic acids, for example oxalic, malonic, succinic, maleic, fumaric, phthalic or terephthalic acids, (c) hydroxycarboxylic acids, for example ascorbic, glycolic, lactic, malic, tartaric or citric acids, (d) amino acids, for example aspartic or glutamic acids, or (e) benzoic acid; or with organic sulfonic acids such as C₁₋₄ alkanesulfonic acids or arylsulfonic acids which may be unsubstituted (e.g., halosubstituted), for example methanesulfonic acid or p-toluenesulfonic acid.

Salts with bases include metal salts such as alkali metal or alkaline earth metal salts, for example sodium, potassium or magnesium salts; or salts with ammonia or an organic amine such as morpholine, thiomorpholine, piperidine, pyrrolidine, a mono-, di- or tri-lower alkyl amine, for example ethylamine, tert-butylamine, diethylamine, diisopropylamine, triethylamine, tributylamine or dimethylpropylamine, or a mono-, di- or tri-(hydroxy lower alkyl) amine, for example monoethanolamine, diethanolamine or triethanolamine.

Alternatively, a prodrug of the compound or a salt of such prodrug can be used. A prodrug is a compound, typically itself having weak or no pharmaceutical activity, that is cleaved, metabolized or otherwise converted in the body of a subject to an active compound, in this case an AT₁ receptor antagonist. Examples of prodrugs are esters, particularly alkanoyl esters and more particularly C₁₋₆ alkanoyl esters. Other examples include carbamates, carbonates, ketals, acetals, phosphates, phosphonates, sulfates and sulfonates, and cilexetil and medoxomil esters as illustrated above in the case of candesartan and olmesartan respectively.

The AT₁ receptor antagonist should be administered according to a treatment regimen effective to reduce growth, invasiveness and/or metastasis of the tumor. One of skill in the art, having the benefit of the present disclosure, will readily and without undue experimentation select a suitable regimen, adjusting it as necessary or desirable in the course of treatment based on clinical response and occurrence of adverse side effects, if any. The term “regimen” in the present context includes dosage amount and frequency, duration of treatment, route of administration and other factors that may be prescribed by the clinician. An appropriate daily dosage amount will in some instances be found in a range already known as an antihypertensive effective dose for the AT₁ receptor antagonist. In other instances, having attention to the seriousness of the disease, it may be desirable to administer a daily dose that is greater than a normal maximum antihypertensive dose. In such cases, it will be especially desirable to monitor the patient for signs of adverse side effects.

Dosages stated herein on a daily or per diem basis should not be interpreted as necessarily being administered on a once daily frequency. Indeed the AT₁ receptor antagonist can be administered at any suitable frequency, for example as determined conventionally by a physician taking into account a number of factors including number, size and invasiveness of tumors, but typically about four times a day, three times a day, twice a day, once a day, every second day, twice a week, once a week, twice a month or once a month. The AT₁ receptor antagonist can alternatively be administered more or less continuously, for example by parenteral infusion in a hospital setting. In some situations a single dose may be administered, but more typically administration is according to a regimen involving repeated dosage over a treatment period. In such a regimen the daily dosage and/or frequency of administration can, if desired, be varied over the course of the treatment period, for example introducing the subject to the compound at a relatively low dose and then increasing the dose in one or more steps until a full dose is reached.

Suitable daily dosage amounts depend on the particular AT₁ receptor antagonist used, as these vary in properties such as receptor affinity, bioavailability, metabolic half-life, etc., and on the route and method of administration. In general, a daily dosage amount should be sufficient to deliver to the target site, i.e., in the present case a breast tumor, a sustained concentration of at least about 30 nM, for example at least about 100 nM, at least about 300 nM or at least about 1 μM, and at most about 1 mM, for example at most about 300 μM, at most about 100 μM or at most about 30 μM, of the administered drug and/or active metabolite(s) thereof. Daily dosage amounts capable of delivering such concentrations when administered systemically to a human patient will typically be about 0.01 to about 100 mg/kg, more typically about 0.02 to about 50 mg/kg, for example about 0.05 to about 25 mg/kg or about 0.1 to about 20 mg/kg. Illustratively, a daily systemic (e.g., oral or parenteral) dose for an adult woman with breast cancer can be about 1 to about 3000 mg, for example about 3 to about 1500 mg or about 5 to about 1000 mg.

In one embodiment, the daily dose is not substantially greater than a dose typically prescribed for treatment of hypertension. According to this embodiment, illustrative doses can be as follows:
candesartan cilexetil: about 4 to about 32 mg/day;
eprosartan: about 150 to about 600 mg/day;
irbesartan: about 75 to about 300 mg/day;
a losartan: about 25 to about 100 mg/day;
olmesartan medoxomil: about 5 to about 40 mg/day;
telmisartan: about 20 to about 80 mg/day;
valsartan: about 40 to about 320 mg/day;
   or, for other AT₁ receptor antagonist drugs, doses therapeutically equivalent thereto. Doses lower than those typically prescribed for treatment of hypertension, for example lower than the doses illustratively shown above, can also be useful in particular cases.

In another embodiment, the daily dose for treatment of breast cancer is higher than a typically prescribed antihypertensive dose, and can be, illustratively, as follows:
candesartan cilexetil: greater than about 32 mg/day;
eprosartan: greater than about 600 mg/day;
irbesartan: greater than about 300 mg/day;
losartan: greater than about 100 mg/day;
olmesartan medoxomil: greater than about 40 mg/day;
telmisartan: greater than about 80 mg/day;
valsartan: greater than about 320 mg/day;
   or, for other AT₁ receptor antagonist drugs, doses therapeutically equivalent thereto; up to about four times, for example about three times or about two times, the maximum typical antihypertensive dose. Even higher doses can be used if tolerated by the patient without an unacceptable degree of adverse side effects.

Where the AT₁ receptor antagonist is administered locally, for example by topical application to the affected area, by injection into a tumor or surrounding tissue, or by surgical implantation, it may be possible to deliver the desired concentration of the drug at the target site by administration of a daily dose that is lower than a systemic dose.

The AT₁ receptor antagonist can be administered in monotherapy, in adjunctive or combination therapy with one or more additional pharmacotherapeutic (including chemotherapeutic) agents, in conjunction with radiation therapy, or as adjuvant therapy to a patient undergoing surgery for breast cancer. For example, the AT₁ receptor antagonist can be administered concomitantly with chemotherapy, radiotherapy and/or surgery to treat the cancer or a secondary tumor derived therefrom.

In one embodiment, the AT₁ receptor antagonist is administered in adjunctive or combination therapy with an anti-hormone drug, which in the present context can comprise an estrogen receptor modulator (more particularly a selective estrogen receptor modulator or SERM), an estrogen receptor antagonist such as fulvestrant, an antiprogestin such as onapristone, and/or an aromatase inhibitor.

Suitable dosages, routes of administration and other aspects of the treatment regimen for the anti-hormone drug will typically be within the normal therapeutic range for the drug when used in monotherapy. However, in some instances it may be possible, when the drug is used in combination therapy with an AT₁ receptor antagonist, to reduce the dose of the anti-hormone drug.

For example, the AT₁ receptor antagonist can be administered in adjunctive or combination therapy with a SERM comprising at least one compound selected from the group consisting of acolbifene, arzoxifene, bazedoxifene, droloxifene, HMR-3339, idoxifene, lasofoxifene, levormeloxifene, ospemifene, raloxifene, tamoxifen, toremifene, pharmaceutically acceptable salts, prodrugs and active metabolites thereof. As indicated hereinabove, combination therapy with an AT₁ receptor antagonist and a SERM can be a good option for treatment of primary infiltrative ductal carcinoma that is ER+ and not SERM-resistant. For SERM-resistant ER+ carcinoma, addition of a SERM to the AT₁ receptor antagonist treatment regimen is less likely to help, but should not be ruled out.

Alternatively, the AT₁ receptor antagonist can be administered in adjunctive or combination therapy with an aromatase inhibitor comprising at least one compound selected from the group consisting of aminoglutethimide, anastrozole, exemestane, fadrozole, formestane, letrozole, vorozole, pharmaceutically acceptable salts, prodrugs and active metabolites thereof.

Combination therapy with an AT₁ receptor antagonist and an aromatase inhibitor or an estrogen receptor antagonist such as fulvestrant can be a good option for treatment of primary infiltrative ductal carcinoma that is ER+, whether or not it is SERM-resistant.

In a further embodiment of the invention, a method is provided for treating SERM-resistant ER+ breast cancer in a female patient. This method comprising administering to the patient an AT₁ receptor antagonist according to a regimen effective to reduce growth, invasiveness and/or metastasis of the tumor.

The method of this embodiment does not necessarily comprise a testing or selection step. The patient to be treated according to the present method can have breast cancer that:
(a) has exhibited inadequate to no beneficial response to prior therapy with a SERM, for example a compound selected from the group consisting of acolbifene, arzoxifene, bazedoxifene, droloxifene, HMR-3339, idoxifene, lasofoxifene, levormeloxifene, ospemifene, raloxifene, tamoxifen, toremifene, pharmaceutically acceptable salts, prodrugs and active metabolites thereof; and/or
(b) has exhibited inadequate to no beneficial response in an assay comprising treatment of tumor cells or a culture thereof derived from the patient with a SERM, in presence of estrogen.

The present method is especially useful where the cancer is ductal carcinoma, more particularly primary infiltrating ductal carcinoma. The AT₁ receptor antagonist, treatment regimen and optional additional drugs used in adjunctive or combination therapy with the AT₁ receptor antagonist can be selected as described above.

A still further embodiment of the invention comprises a therapeutic combination comprising an AT₁ receptor antagonist and an aromatase inhibitor in amounts effective in combination to reduce growth, invasiveness and/or metastasis of a breast tumor, in particular embodiments an ER+ breast tumor, for example a SERM-resistant breast tumor. Suitable absolute and relative amounts of the AT₁ receptor antagonist and the aromatase inhibitor will be based on therapeutically effective dosage amounts of each, but in some instances it will be found possible to reduce the dosage amount of one or other component of the therapeutic combination without loss of efficacy.

Illustratively, the AT₁ receptor antagonist can be selected from A-81282, A-81988, BMS-184,698, candesartan, CGP-49870, CI-996, CL-329,167, CP-161418, D-8731, DMP-581, DMP-811, DuP-532, E-4177, EMD-66397, eprosartan, EXP-408, EXP-970, EXP-3892, EXP-6803, EXP-7711, GA-0050, GR-138,950, HN-65,021, irbesartan, KRH-594, KT-3671, KW-3433, L-158,809, L-158,978, L-159,282, L-159,686, L-159,689, L-159,874, L-161,177, L-161,816, L-162,154, L-162,234, L-162,441, L-163,007, L-163,017, LF-7-0156, losartan, LR-B-081, LY-285,434, ME-3221, MK-996, olmesartan, PD-123,177, PD-123,319, PD-150,304, RWJ-38970, RWJ-46458, saprisartan, SC-48742, SC-50560, SC-51316, SC-51895, SC-52458, SL-910,102, TA-606, TAK-536, tasosartan, telmisartan, U-96,849, UP-269-6, UP-27,522, UR-7198, valsartan, WAY-126,227, WK-1492, XH-148, XR-510, YM-358, YM-31,472, ZD-8731, zolarsartan and pharmaceutically acceptable salts, prodrugs and active metabolites thereof. Illustratively, the aromatase inhibitor can be selected from aminoglutethimide, anastrozole, exemestane, fadrozole, formestane, letrozole, vorozole, pharmaceutically acceptable salts, prodrugs and active metabolites thereof. Optionally more than one AT₁ receptor antagonist and/or more than one aromatase inhibitor can be present in the combination.

The components of the therapeutic combination of the present embodiment can be present in separate pharmaceutical compositions or in a single pharmaceutical composition. Such a single pharmaceutical composition, comprising an AT₁ receptor antagonist, an aromatase inhibitor and at least one pharmaceutically acceptable excipient, is a further embodiment of the present invention.

A method for treating a breast tumor in a female patient, comprising administering to the patient a therapeutic combination comprising an AT₁ receptor antagonist and an aromatase inhibitor, is a still further embodiment of the invention.

The method is particularly appropriate for treating an ER+ breast tumor, whether SERM-responsive or SERM-resistant. The tumor can be a ductal or lobular carcinoma; in a particular embodiment the tumor is primary infiltrating ductal carcinoma. The combination can be administered separately or together; if together, the components of the combination can be administered in separate pharmaceutical compositions or in a single pharmaceutical composition.

A kit comprising (a) a first container containing a first pharmaceutical composition comprising at least one unit dosage amount of an AT₁ receptor antagonist and (b) a second container containing a second pharmaceutical composition comprising at least one dosage amount of an aromatase inhibitor is a still further embodiment of the invention. Such a kit can further comprise means for communicating information or directions on administration of the first and second compositions to a female patient having breast cancer, more particularly ER+ breast cancer. Examples of such communicating means include printed information, for example on a label, brochure, package insert or advertisement; information in electronic form, for example on a web page; or information in audiovisual form, for example on audiotape, videotape or DVD. The information can be directed primarily to the patient herself, or to a caregiver of the patient, or to the patient's physician.

A still further embodiment of the invention comprises a therapeutic combination comprising an AT₁ receptor antagonist and an estrogen receptor modulator or antagonist in amounts effective in combination to reduce growth, invasiveness and/or metastasis of a breast tumor, in particular embodiments an ER+ breast tumor. Suitable absolute and relative amounts of the AT₁ receptor antagonist and the estrogen receptor modulator or antagonist will be based on therapeutically effective dosage amounts of each, but in some instances it will be found possible to reduce the dosage amount of one or other component of the therapeutic combination without loss of efficacy.

Illustratively, the AT₁ receptor antagonist can be selected from A-81282, A-81988, BMS-184,698, candesartan, CGP-49870, CI-996, CL-329,167, CP-161418, D-8731, DMP-581, DMP-811, DuP-532, E-4177, EMD-66397, eprosartan, EXP-408, EXP-970, EXP-3892, EXP-6803, EXP-7711, GA-0050, GR-138,950, HN-65,021, irbesartan, KRH-594, KT-3671, KW-3433, L-158,809, L-158,978, L-159,282, L-159,686, L-159,689, L-159,874, L-161,177, L-161,816, L-162,154, L-162,234, L-162,441, L-163,007, L-163,017, LF-7-0156, losartan, LR-B-081, LY-285,434, ME-3221, MK-996, olmesartan, PD-123,177, PD-123,319, PD-150,304, RWJ-38970, RWJ-46458, saprisartan, SC-48742, SC-50560, SC-51316, SC-51895, SC-52458, SL-910,102, TA-606, TAK-536, tasosartan, telmisartan, U-96,849, UP-269-6, UP-27,522, UR-7198, valsartan, WAY-126,227, WK-1492, XH-148, XR-510, YM-358, YM-31,472, ZD-8731, zolarsartan and pharmaceutically acceptable salts, prodrugs and active metabolites thereof. Illustratively, an estrogen receptor modulator, more particularly a SERM, can be selected from acolbifene, arzoxifene, bazedoxifene, droloxifene, HMR-3339, idoxifene, lasofoxifene, levormeloxifene, ospemifene, raloxifene, tamoxifen, toremifene, pharmaceutically acceptable salts, prodrugs and active metabolites thereof. Illustratively, the estrogen receptor antagonist fulvestrant or a pharmaceutically acceptable salt, prodrug or active metabolite thereof can be present in the combination. Optionally more than one AT₁ receptor antagonist and/or more than one estrogen receptor modulator and/or antagonist can be present in the combination.

The components of the therapeutic combination of the present embodiment can again be present in separate pharmaceutical compositions or in a single pharmaceutical composition. Such a single pharmaceutical composition, comprising an AT₁ receptor antagonist, an estrogen receptor modulator or antagonist and at least one pharmaceutically acceptable excipient, is a further embodiment of the present invention.

A method for treating a breast tumor in a female patient, comprising administering to the patient a therapeutic combination comprising an AT₁ receptor antagonist and an estrogen receptor modulator or antagonist, is a still further embodiment of the invention.

Such a tumor will normally be ER+ and can be SERM-responsive or SERM-resistant; however, if it is SERM-resistant an estrogen receptor antagonist, such as fulvestrant or a pharmaceutically acceptable salt, prodrug or active metabolite thereof, will in some situations be a better option for the therapeutic combination than a SERM. The tumor can be a ductal or lobular carcinoma; in a particular embodiment the tumor is primary infiltrating ductal carcinoma. The combination can again be administered separately or together; if together, the components of the combination can be administered in separate pharmaceutical compositions or in a single pharmaceutical composition.

A kit comprising (a) a first container containing a first pharmaceutical composition comprising at least one unit dosage amount of an AT₁ receptor antagonist and (b) a second container containing a second pharmaceutical composition comprising at least one dosage amount of an estrogen receptor modulator or antagonist is a still further embodiment of the invention. Such a kit can further comprise means for communicating information or directions on administration of the first and second compositions to a female patient having ER+ breast cancer. Examples of such communicating means are as described hereinabove.

Methods of the invention can comprise administration of compounds as described above by any appropriate route, which can result in local or systemic delivery, or both. Examples of primarily local administration methods suitable in practice of the invention include topical application, local injection and surgical implantation. Examples of primarily systemic administration methods suitable in practice of the invention include oral, rectal, nasal, transmucosal, intrapulmonary, intravenous, intraperitoneal, intramuscular, subcutaneous, intradermal and transdermal administration.

While it can be possible to administer the compound, or a salt or prodrug thereof unformulated as active pharmaceutical ingredient (API) alone, it will generally be found preferable to administer the API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below.

USIP, ed. (2005) Remington: The Science and Practice of Pharmacy, 21st ed., Lippincott, Williams & Wilkins.

Allen et al. (2004) Ansel's Pharmaceutical Dosage Forms and Drug Delivery Systems, 8th ed., Lippincott, Williams & Wilkins.

Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present invention include, without limitation, solutions, suspensions, powders, granules, tablets, capsules, pills, lozenges, chews, creams, ointments, gels, liposome preparations, nanoparticulate preparations, injectable preparations, enemas, suppositories, inhalable powders, sprayable liquids, aerosols, patches, depots and implants.

Illustratively, in a liquid formulation suitable, for example, for parenteral, intranasal or oral delivery, the API can be present in solution or suspension, or in some other form of dispersion, in a liquid medium that comprises a diluent such as water. Additional excipients that can be present in such a formulation include a tonicifying agent, a buffer (e.g., a tris, phosphate, imidazole or bicarbonate buffer), a dispersing or suspending agent and/or a preservative. Such a formulation can contain micro- or nanoparticulates, micelles and/or liposomes. A parenteral formulation can be prepared in dry reconstitutable form, requiring addition of a liquid carrier such as water or saline prior to administration by injection.

For rectal delivery, the API can be present in dispersed form in a suitable liquid (e.g. as an enema), semi-solid (e.g., as a cream or ointment) or solid (e.g., as a suppository) medium. The medium can be hydrophilic or lipophilic.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 99%, for example about 10% to about 85%, or about 20% to about 80%, by weight of the composition. The diluent or diluents selected preferably exhibit suitable flow properties and, where tablets are desired, compressibility.

Lactose, microcrystalline cellulose and starch, either individually or in combination, are particularly useful diluents.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC or hypromellose), hydroxypropyl-cellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone), for example povidone K-15, K-30 and K-29/32; polyacrylic acids (carbomers); polymethacrylates; and the like. One or more binding agents and/or adhesives, if present, typically constitute in total about 0.5% to about 25%, for example about 0.75% to about 15%, or about 1% to about 10%, by weight of the composition.

Povidone is a particularly useful binding agent for tablet formulations, and, if present, typically constitutes about 0.5% to about 15%, for example about 1% to about 10%, or about 2% to about 8%, by weight of the composition.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.2% to about 10%, or about 0.2% to about 5%, by weight of the composition.

Croscarmellose sodium and crospovidone, either individually or in combination, are particularly useful disintegrants for tablet or capsule formulations, and, if present, typically constitute in total about 0.2% to about 10%, for example about 0.5% to about 7%, or about 1% to about 5%, by weight of the composition.

Wetting agents, if present, are normally selected to maintain the drug or drugs in close association with water, a condition that is believed to improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; sorbitan esters, for example sorbitan monolaurate, sorbitan monooleate, sorbitan monopannitate and sorbitan monostearate; tyloxapol; and the like. One or more wetting agents, if present, typically constitute in total about 0.25% to about 15%, preferably about 0.4% to about 10%, and more preferably about 0.5% to about 5%, by weight of the composition.

Wetting agents that are anionic surfactants are particularly useful. Illustratively, sodium lauryl sulfate, if present, typically constitutes about 0.25% to about 7%, for example about 0.4% to about 4%, or about 0.5% to about 2%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 8%, or about 0.2% to about 5%, by weight of the composition. Magnesium stearate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.1% to about 10%, for example about 0.1% to about 5%, or about 0.1% to about 2%, by weight of the composition.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.1% to about 10%, for example about 0.1% to about 5%, or about 0.1% to about 2%, by weight of the composition.

Talc and colloidal silicon dioxide, either individually or in combination, are particularly useful anti-adherents and glidants.

Other excipients such as buffering agents, stabilizers, antioxidants, antimicrobials, colorants, flavors and sweeteners are known in the pharmaceutical art and can be used. Tablets can be uncoated or can comprise a core that is coated, for example with a nonfunctional film or a release-modifying or enteric coating. Capsules can have hard or soft shells comprising, for example, gelatin and/or HPMC, optionally together with one or more plasticizers.

A pharmaceutical composition useful herein typically contains the compound or salt or prodrug thereof in an amount of about 1% to about 99%, more typically about 5% to about 90% or about 10% to about 60%, by weight of the composition. A unit dosage form such as a tablet or capsule can conveniently contain an amount of the compound providing a single dose, although where the dose required is large it may be necessary or desirable to administer a plurality of dosage forms as a single dose. Illustratively, a unit dosage form can comprise the compound in an amount of about 1 to about 800 mg, for example about 5 to about 750 mg or about 10 to about 600 mg.

For oral administration, conventional unit dosage forms such as tablets or capsules, including AT₁ receptor antagonist dosage forms commercially available for treatment of hypertension, are generally suitable for use according to the present methods. Thus, for example, the dosage forms sold under the trade names Atacand® (candesartan cilexetil), Avapro® (irbesartan), Benicar® (olmesartan medoxomil), Cozaar® (losartan), Diovan® (valsartan), Micardis® (telmisartan) and Teveten® (eprosartan mesylate) are useful herein. Alternatively, dosage forms of these and other AT₁ receptor antagonist drugs more specifically adapted to the present use can be developed.

Compounds useful herein can alternatively be delivered to a target site by surgical implantation into an area affected by a tumor, with or without surgical excision of the tumor. Implantable compositions can comprise an AT₁ receptor antagonist in a biodegradable polymer matrix. A method for delivery of an anticancer drug after surgical resection is described, for example, by Fleming & Saltzman (2002) Clin. Pharmacokinetics 41:403-419, and can be adapted to treatment of breast cancer. Implantation therapy with an AT₁ receptor antagonist, optionally together with one or more additional drugs, can be combined, if desired, with one or more of surgery, radiotherapy, chemotherapy and immunotherapy. Implants typically provide sustained release of the drug over an extended period, for example about 7 days to about 100 days.

A biodegradable polymer useful in preparation of an implantable composition useful herein can comprise any polymer or copolymer that, upon degradation, can dissolve in interstitial fluid without unacceptable adverse effect or toxicity. Certain polymers or monomers from which such polymer are synthesized are approved by the U.S. Food and Drug Administration (FDA) for implantation into humans. A copolymer comprising monomers having different dissolution properties can provide control of dynamics of degradation, for example by increasing the proportion of one monomer over another to control rate of dissolution.

Other delivery systems providing extended release of a drug are also available and adaptable to use with an AT₁ receptor antagonist. Such systems include, for example, nanoparticulate systems that can provide sustained and targeted delivery of a drug within or in close proximity to a tumor.

The present invention derives in part from unexpected findings with regard to level of expression of AT₁ receptor mRNA and/or protein in breast tissue affected by ER+ versus ER− carcinoma, especially infiltrative ductal carcinoma. “Overexpression” or “up-regulation” herein typically means that the receptor, or mRNA encoding the receptor, is expressed in a particular tissue at least about 20% more highly than in a comparison tissue such as normal breast tissue. In various embodiments, AT₁ receptor mRNA and/or protein expression in tissue of a subject to be selected for AT₁ receptor antagonist therapy is at least about 50% higher, for example at least about 100% (about 2-fold) higher than in normal breast tissue.

The present methods are directed to selection, screening and/or treatment of female patients. Patients herein are generally human patients, but it will be understood that the methods are adaptable to other species, including animal models for human disease and to animals requiring veterinary care.

### EXAMPLES

The following Examples illustrate the invention using data mining, computational biology, cell proliferation and in vivo tumor growth experiments to demonstrate utility and efficacy of methods of the invention.

As determined by gene expression profiling (Example 1), AT₁ receptors are shown to be overexpressed in ER+, but not in ER−, infiltrating ductal carcinomas of the breast relative to normal breast tissue. Proliferation of ER+, but not ER−, ductal carcinoma cells in vitro is shown to be stimulated by Ang II (Example 2), and blockade of Ang II signaling by a variety of AT₁ receptor antagonists is shown to inhibit such Ang II-induced proliferation (Examples 3-5). In a series of breast cancer cell lines (Example 6), most are shown to express AT₁ receptor protein but stimulation of cell proliferation by Ang II is seen only in lines that also express ERα protein (Example 7). Development of cell lines transfected with a luciferase gene (Example 8) enables in vivo confirmation of efficacy of AT₁ receptor antagonists on ER+ by comparison with ER− tumors in mice, for example using hollow fiber assay methods (Example 9). Clinical applications utilizing ER+/ER− patient stratification made possible by the present invention are illustrated in Examples 10 and 11.

### Example 1

### AT₁ Receptor mRNA Expression

AT₁ receptor mRNA expression was quantified in human tissues, using the BioExpress® System of Gene Logic Inc. This system includes mRNA expression data from about 18,000 samples, of which about 90% are from human tissues, comprising both normal and diseased samples from about 435 disease states. In brief, human tissue samples, either from surgical biopsy or post-mortem removal, were processed for mRNA expression profile analysis using Affymetrix GeneChips®. Each tissue sample was examined by a board-certified pathologist to confirm pathological diagnoses. RNA isolation, cDNA synthesis, cRNA amplification and labeling, hybridizations, and signal normalization were carried out using standard Affymetrix protocols. Computational analysis was performed using Genesis Enterprise System® Software and the Ascenta® software system (Gene Logic Inc).

AT₁ receptor expression data from two probes based on different parts of the AT₁ receptor nucleotide sequence are summarized in Table 1. N=number of tissue samples; SD=standard deviation.

TABLE 1
Summary of AT₁ receptor mRNA expression data

| | Probe 1 | | Probe 2 | |
|---|---|---|---|---|
| | | av. fold | | av. fold |
| | | change vs. | | change vs. |
| Breast tissue | mean | normal | mean | normal |
| infiltrating ductal carcinoma, | 580 | 3.98 | 219 | 4.43 |
| primary, ER+ PR+ | | | | |
| infiltrating ductal carcinoma, | 544 | 3.74 | 206 | 4.18 |
| primary, ER+ | | | | |
| infiltrating ductal carcinoma, | 490 | 3.37 | 186 | 3.77 |
| primary, ER+ PR− | | | | |
| infiltrating ductal carcinoma, | 44 | 0.30 | 21 | 0.42 |
| primary, ER− | | | | |
| infiltrating ductal carcinoma, | 35 | 0.24 | 19 | 0.39 |
| primary, ER− PR− | | | | |
| infiltrating ductal carcinoma, | 529 | 3.63 | 210 | 4.25 |
| primary, low stage, ER+ | | | | |
| infiltrating ductal carcinoma, | 26 | 0.18 | 12 | 0.25 |
| primary, low stage, ER− | | | | |
| infiltrating ductal carcinoma, | 609 | 4.19 | 254 | 5.14 |
| primary, low stage, PR+ | | | | |
| infiltrating ductal carcinoma, | 231 | 1.58 | 85 | 1.73 |
| primary | | | | |
| infiltrating ductal carcinoma, | 271 | 1.86 | 120 | 2.43 |
| primary, Her2-neu+ | | | | |
| infiltrating ductal carcinoma, | 653 | 4.48 | 251 | 5.09 |
| primary, Her2-neu− | | | | |
| infiltrating ductal carcinoma, | 62 | 0.43 | 27 | 0.56 |
| primary, p53+ | | | | |
| infiltrating ductal carcinoma, | 148 | 1.01 | 50 | 1.01 |
| primary, p53− | | | | |
| infiltrating ductal carcinoma, | 333 | 2.29 | 134 | 2.71 |
| primary, smoking history | | | | |
| infiltrating ductal carcinoma, | 118 | 0.81 | 38 | 0.77 |
| primary, stage I | | | | |
| infiltrating ductal carcinoma, | 260 | 1.78 | 98 | 1.99 |
| primary, stage II | | | | |
| infiltrating ductal carcinoma, | 46 | 0.32 | 28 | 0.56 |
| primary, stage III | | | | |
| intraductal carcinoma | 78 | 0.53 | 33 | 0.67 |
| infiltrating mixed ductal and | 44 | 0.30 | 23 | 0.46 |
| lobular, primary | | | | |
| infiltrating lobular carcinoma, | 68 | 0.46 | 32 | 0.66 |
| primary, ER+ | | | | |
| infiltrating lobular carcinoma, | 78 | 0.54 | 37 | 0.75 |
| primary, PR+ | | | | |
| infiltrating lobular carcinoma, | 43 | 0.30 | 22 | 0.44 |
| primary, Her2-neu + | | | | |
| infiltrating lobular carcinoma, | 815 | 5.60 | 164 | 3.33 |
| primary, stage I | | | | |
| infiltrating lobular carcinoma, | 326 | 2.24 | 118 | 2.39 |
| primary, stage III | | | | |
| infiltrating lobular carcinoma, | 110 | 0.76 | 36 | 0.73 |
| primary, smoking history | | | | |
| infiltrating lobular carcinoma, | 278 | 1.91 | 68 | 1.37 |
| primary, no smoking history | | | | |
| mucinous carcinoma, | 492 | 3.38 | 170 | 3.45 |
| primary | | | | |
| phyllodes tumor | 129 | 0.89 | 62 | 1.26 |
| fibrocystic disease | 151 | 1.04 | 54 | 1.09 |
| fibroadenoma | 86 | 0.59 | 37 | 0.76 |
| normal | 146 | 1.00 | 49 | 1.00 |
| normal, | 134 | 0.92 | 43 | 0.87 |
| smoking history | | | | |
| normal, | 153 | 1.05 | 49 | 0.98 |
| no smoking history | | | | |
| normal, | 132 | 0.91 | 49 | 0.99 |
| taking levothyroxine | | | | |
| normal, | 160 | 1.10 | 46 | 0.92 |
| not taking levothyroxine | | | | |
| normal, primary malignancy | 125 | 0.86 | 42 | 0.86 |
| elsewhere in breast | | | | |
| normal, no disease | 197 | 1.35 | 68 | 1.38 |
| elsewhere in breast | | | | |

Overall, it can be seen from Table 1 that relative AT₁ receptor expression levels in various disease states with respect to ER and PR status were, from highest to lowest:
infiltrating ductal carcinoma, primary, ER+ PR+
infiltrating ductal carcinoma, primary, ER+ PR−
   normal
   infiltrating lobular carcinoma, primary, PR+
   infiltrating lobular carcinoma, primary, ER+
   infiltrating ductal carcinoma, primary, ER−
infiltrating ductal carcinoma, primary, ER−PR−

It can further be seen from Table 1 that relative AT₁ receptor expression levels in various stages of infiltrative ductal carcinoma, without regard to ER or PR status, were, from highest to lowest:
primary, stage II
   normal
primary, stage I
primary, stage III

Explicit t-test comparisons of mRNA expression levels in pairs of samples (one of each pair identified as “experiment” and the other as “control”) were made using DiffX analysis. Sample comparisons are presented in Table 2.

TABLE 2
Sample comparisons of AT₁ receptor mRNA expression by DiffX analysis

| | | | Fold | |
|---|---|---|---|---|
| Experiment | Control | Probe | change | P |
| infiltrating ductal carcinoma, | infiltrating ductal carcinoma, | 1 | 16.49 | 0.0007 |
| primary, ER+ PR+ | primary, ER− PR− | | | |
| infiltrating ductal carcinoma, | infiltrating ductal carcinoma, | 2 | 11.49 | 0.0011 |
| primary, ER+ PR+ | primary, ER− PR− | | | |
| infiltrating ductal carcinoma, | infiltrating ductal carcinoma, | 1 | 12.15 | 0.0001 |
| primary, ER+ | primary, ER− | | | |
| infiltrating ductal carcinoma, | infiltrating ductal carcinoma, | 2 | 9.55 | 0.0002 |
| primary, ER+ | primary, ER− | | | |
| infiltrating ductal carcinoma, | infiltrating ductal carcinoma, | 1 | 23.08 | 0.025 |
| primary, low stage, ER+ | primary, low stage, ER− | | | |
| infiltrating ductal carcinoma, | infiltrating ductal carcinoma, | 2 | 16.86 | 0.022 |
| primary, low stage, ER+ | primary, low stage, ER− | | | |
| infiltrating ductal carcinoma, | infiltrating lobular carcinoma, | 1 | 8.05 | 0.0002 |
| primary, ER+ | primary, ER+ | | | |
| infiltrating ductal carcinoma, | infiltrating lobular carcinoma, | 2 | 6.37 | 0.0005 |
| primary, PR+ | primary, PR+ | | | |
| infiltrating ductal carcinoma, | infiltrating lobular carcinoma, | 1 | 6.25 | 0.018 |
| primary, Her2-neu+ | primary, Her2-neu+ | | | |
| infiltrating ductal carcinoma, | infiltrating ductal carcinoma, | 1 | 2.18 | 0.030 |
| primary, stage II | primary, stage I | | | |
| infiltrating ductal carcinoma, | infiltrating ductal carcinoma, | 1 | 0.39 | 0.039 |
| primary, stage III | primary, stage I | | | |

Thus, surprisingly, not only is AT₁ receptor mRNA strongly up-regulated in ER+infiltrating ductal carcinoma, but it is strongly down-regulated in ER− infiltrating ductal carcinoma. This finding suggests for the first time that an AT₁ receptor antagonist is likely to provide little benefit in treatment of invasive ER− breast cancer. Additionally, AT₁ receptor mRNA is much more strongly expressed in infiltrating ductal carcinoma than in infiltrating lobular carcinoma, even in the case of ER+ or PR+ lobular carcinoma.

### Example 2

### Ang II-Induced Cell Proliferation

Various methods of measuring cell proliferation are described in the publications individually cited below and incorporated herein by reference.

Solly et al. (2004) Assay Drug Dev. Technol. 2(4):363-372.

Giaever & Keese (1984) Proc. Natl. Acad. Sci. 81(12):3761-3764.

Mitra et al. (1991) Biotechniques 11(4):504-510.

Xiao & Luong (2003) Biotechnol. Prog. 19(3):1000-1005.

Unless otherwise indicated, cell proliferation assays described in Examples 2-5 were performed using a Real-Time Cell Electronic Sensing (RT-CES™ 96×) instrument from ACEA Bioscience (San Diego, Calif.). This instrument utilizes an electronic readout (impedance) to non-invasively quantify adherent cell proliferation and viability in real time.

Cells were seeded in 96-well microtiter plates containing microelectronic sensor arrays (96E plates; ACEA). Cells were maintained in RPMI 1640 (Invitrogen, Carlsbad, Calif.) supplemented with 10% fetal bovine serum (FBS) (Hyclone, Logan, Utah) and were cultured at 37° C. in a humidified atmosphere containing 5% CO₂. For proliferation assays, cells were seeded (20,000 cells/well), allowed to attach and grown overnight in standard growth medium. Cells were then serum-starved for 8 hours prior to the proliferation assay in presence or absence of test compound(s). The proliferation response was continuously monitored by measuring the impedance change in each well for the indicated number of hours. Data are expressed as cell index (CI) change relative to time in culture, reflecting measured changes in electrical impedance. Each value shown is an average of 6 wells. Where shown, statistical analysis was performed by calculation of standard deviation and P values were generated by a Student's two-tailed t-test.

Experiments were performed to test the hypothesis that ER+ ductal carcinoma cells would be more responsive to Ang II-induced stimulation than ER− cells.

T47D (American Type Culture Collection, Manassas, Va. (ATCC) cat. HTB-133) is an ER+ cell line derived from human mammary gland ductal carcinoma. Other ER+ cell lines used in studies reported herein include ZR-75 and HCC70. HCC1143 (ATCC cat. CRL-2321) is an ER− cell line also derived from human mammary gland ductal carcinoma.

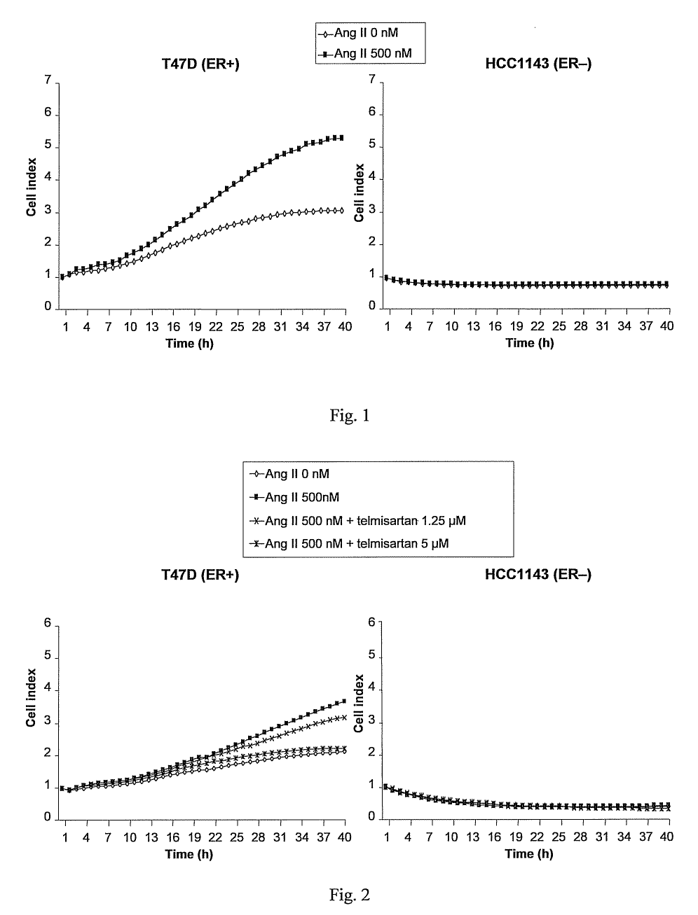

Following the 8 hour starvation phase, either Ang II (Sigma-Aldrich), 500 nM, or vehicle control was added to the cell culture. The results, shown in FIG. 1, show that Ang II significantly stimulated growth of the ER+ cell line T47D, but had no effect on the ER− cell line HCC1143. Ang II also stimulated proliferation of ER+ cell lines HCC70, by approximately 30%, and ZR-75, by approximately 35%, relative to vehicle control (data not shown).

### Example 3

### Inhibition of Ang II-Induced Cell Proliferation by Telmisartan

A cell proliferation assay procedure was followed as described in Example 2. Following the 8 hour starvation phase, either Ang II (Sigma-Aldrich), 500 nM, with or without the AT₁ receptor antagonist telmisartan, 1.25 μM or 5 μM, or vehicle control was added to the cell culture. The results, presented in FIG. 2, show that telmisartan significantly inhibited Ang II-induced growth of the ER+ cell line T47D in a concentration dependent manner. No effects of Ang II or telmisartan were seen in the ER− cell line HCC1143.

### Example 4

### Inhibition of Ang II-Induced Cell Proliferation by Candesartan

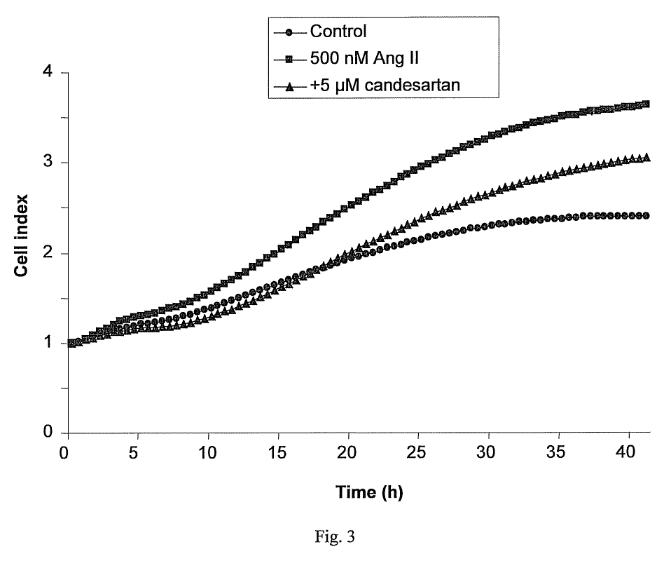

A cell proliferation assay procedure was followed as described in Example 2. Following the 8 hour starvation phase, either Ang II (Sigma-Aldrich), 500 nM, with or without the AT₁ receptor antagonist candesartan, 5 μM, or vehicle control was added to the cell culture. The results, presented in FIG. 3, show that candesartan significantly inhibited Ang II-induced growth of the ER+ cell line T47D. No effects of Ang II or candesartan were seen in the ER− cell line HCC1143 (data not shown).

### Example 5

### Inhibition of Ang II-Induced Cell Proliferation by Irbesartan

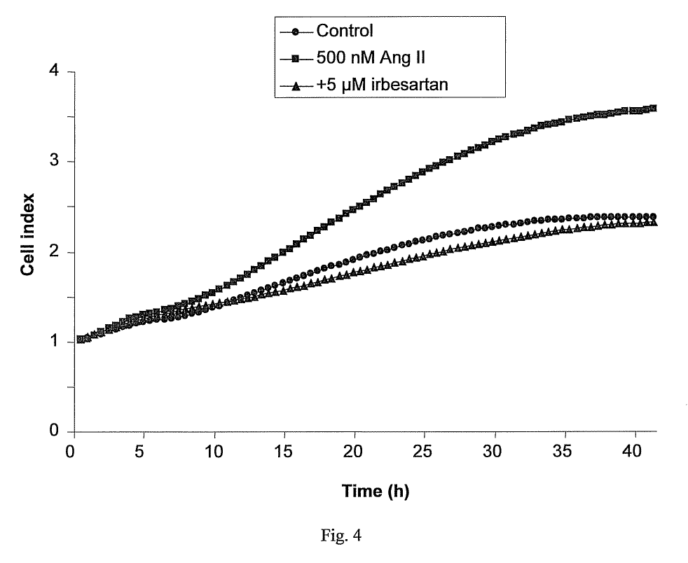

A cell proliferation assay procedure was followed as described in Example 2. Following the 8 hour starvation phase, either Ang II (Sigma-Aldrich), 500 nM, with or without the AT₁ receptor antagonist irbesartan, 5 μM, or vehicle control was added to the cell culture. The results, shown in FIG. 4, show that irbesartan significantly inhibited Ang II-induced growth of the ER+ cell line T47D. No effects of Ang II or irbesartan were seen in the ER− cell line HCC1143 (data not shown).

### Example 6

### ER and AT₁ Receptor Antigen Expression in Various Ductal Carcinoma Cell Lines

Human breast carcinoma cell lines were collected in ice-cold RIPA buffer (Sigma) containing protease inhibitors to prevent proteolytic degradation. Samples were lysed on ice for 30 minutes and cleared by centrifugation at 10,000 rpm for 10 minutes. Total protein in the supernatant was estimated using a BCA™ assay kit (Pierce, Rockford, Ill.). Samples were resolved on a 10% Nupage gel (Invitrogen) at 50 μg and 60 μg total protein/lane for ERα and AT₁ receptor, respectively. Seeblue and Magicmark molecular weight markers (Invitrogen) were used for estimation of molecular size. Proteins were transferred to a PVDF membrane and probed with anti-ERα (Affity Bioreagents—1:1000) or anti-AT₁ receptor (Fitzgerald —1:1000) antibody overnight at 4° C. After washing off the unbound primary antibody with PBST, membranes were incubated for 2 hours with horseradish peroxidase-conjugated anti-rabbit (1:10,000) or alkaline phosphatase-conjugated anti-mouse (1:1000) secondary antibodies for ERα and AT₁ receptor, respectively. After extensive washing with PBST the membranes were developed using SigmaFast NBT/BCIP developing solution or Amersham ECL Western blotting detection kit for ERα and AT₁ receptor, respectively. The results, shown in Table 3, were determined to be positive or negative by visual inspection of the gel bands.

TABLE 3
Antigen expression for ERα and AT₁ receptor in a panel of human breast
ductal carcinoma cell lines

| Cell line | ERα | AT₁ |
|---|---|---|
| T47D | positive | positive |
| ZR-75 | positive | positive |
| HCC70 | positive | positive |
| HCC1395 | positive | positive |
| HCC1143 | negative | positive |
| HCC1954 | negative | positive |
| HCC1937 | negative | positive |
| HCC38 | n.d. | negative |

n.d. = not determined

### Example 7

### Correlation of Cell Proliferation Response to Ang II with ER and AT₁ Receptor Antigen Expression in Various Ductal Carcinoma Cell Lines

The data presented in Example 2 for the T47D (ER+) and HCC1143 (ER−) cell lines preliminarily indicate that expression of both ERα and AT₁ receptor are a requisite for responsiveness to Ang II-induced cell proliferation. To provide confirmation, additional experiments were performed to profile each of a panel of cell lines for their response to Ang II. Cell proliferation experiments were performed as described in Example 2 using identical assay methods and instrumentation. As shown in Table 4, data for these studies indicate a strong correlation between Ang II-induced cell proliferation response and expression of both ERα and AT₁ receptor, although cell line HCC1395 represents an outlier in this analysis.

TABLE 4
Ang II-induced proliferation in relation to ERα and
AT₁ receptor expression

| | Ang II proliferation | | |
|---|---|---|---|
| Cell line | response | ERα | AT₁ |
| T47D | positive | + | + |
| ZR-75 | positive | + | + |
| HCC70 | positive | + | + |
| HCC1395 | negative | + | + |
| HCC1143 | negative | − | + |
| HCC1954 | negative | − | + |
| HCC1937 | negative | − | + |
| HCC38 | negative | n.d. | − |

n.d. = not determined

### Example 8

### Generation of Breast Cancer Cell Lines Constitutively Expressing Luciferase Activity

The results presented above indicate that the cell line T47D (ER+) but not HCC1143 (ER−) is responsive to Ang II-induced proliferation and that this response can be inhibited with AT₁ receptor antagonists such as irbesartan, candesartan and telmisartan. To confirm this effect in vivo, appropriate bioluminescent cell lines can be generated for hollow fiber tube implantation in mice. This approach allows seeding of the implanted hollow fiber tubes with cells of interest which can then be studied using bioluminescent imaging to detect alterations in growth (proliferative capacity) upon exposure to AT₁ receptor antagonists. Imaging capabilities can be provided, for example, by bioluminescence imaging technology such as the Xenogen Bio IVIS 200 platform.

The SV40 promoter from pGL3.0 control vector (Promega, Madison, Wis., cat. E1741) was cut with NheI and HindIII restriction enzymes and the resulting fragment was ligated into pGL4.17 (Promega, cat. E6721). The resulting construct (pGL-SV40luc) was then characterized using restriction enzyme digestion to confirm the correct insert size. This vector construct also contains in the plasmid backbone DNA encoding aminoglycoside phosphotransferase, which provides a selectable marker for transfected cells when grown in the presence of geneticin.

Suitable methods for molecular cloning are described, for example in Sambrook & Russell, eds. (2001) Molecular Cloning: A Laboratory Manual, 3rd ed.; Cold Spring Harbor Laboratory Press, Cold Spring Harbor, N.Y.

T47D and HCC1143 cells were seeded in 6-well plates at a density of 2.0×10⁶ cells/well and allowed to attach overnight. Linearization of vector pGL-SV40luc was achieved by digesting the purified DNA with Sal I restriction enzyme. Purified linear DNA (1 μg/well) was transfected into T47D and HCC1143 cells using Fugene 6 reagent (Roche, cat. 11 814 443 001) according to the manufacturer's recommendations. The next day, transfection media was removed from the wells and the transfected cells were washed with PBS, trypsinized and placed into 150 cm dishes at various cell densities. A portion of the cells were used in a luciferase assay to confirm vector function and transfection efficiency. Specifically, a luciferase activity assay was performed using premixed Bright-Glo luciferase assay reagent (Promega, cat. E6250). After 24 hours the growth media was removed and replaced with fresh growth media containing 600 μg/ml geneticin. The growth media was changed every 2-3 days while maintaining the same concentration of geneticin. On days 11-20 post-transfection, the culture was monitored for formation of single colony clones and when identified these single colonies were harvested and transferred into 96 flat-bottom well TC plates. Cells that did not form single colonies were pooled to obtain a mixed population of positively selected cells. The single colony clones were expanded using standard growth conditions to generate sufficient cells for analysis. Luciferase assays were then performed to compare the luciferase activity of both individual clones and the pooled cell populations.

At least one clone from cell line HCC1143, herein referred to as HCC1143-LUC, was found to exhibit a high level of constitutive luciferase activity. In a similar manner, at least one clone from cell line T47D, herein referred to as T47D-LUC, was found to exhibit a high level of constitutive luciferase activity. Finally, the pooled stable lines were analyzed for luciferase expression and both the pooled HCC1143-LUC and the pooled T47D-LUC clones were found to exhibit high levels of constitutive luciferase activity relative to empty vector transfected cells.

### Example 9

### In Vivo Hollow Fiber Assay

As described above, both clonal and pooled ER+ (T47D-LUC) and ER−(HCC1143-LUC) cell lines have been developed with constitutive expression of a luciferase reporter.

Luciferase-expressing cells prepared as described above are injected into polyvinylidene fluoride hollow fibers (50 kDa MW cutoff). Nutrients and test compounds are able to diffuse though the hollow fibers while cells are sequestered inside the fibers. Experiments are first performed in vitro to assess the ability of the cells to survive and grow within the hollow fibers submerged in tissue culture medium. Hollow fibers are imaged daily using a highly light-sensitive CCD chip camera system to observe cell survival and growth as a function of luciferase expression.

Once growth parameters have been observed in vitro, cell-filled hollow fibers are inserted beneath the skin of nude or nude/scid mice. Each animal serves as its own control with one hollow fiber containing the ER+ and the other the ER− cell line. Groups of mice are dosed with either vehicle control or AT₁ receptor antagonists, optionally in combination with anti-estrogen compounds (e.g., SERMs or aromatase inhibitors). Mice can be imaged daily for up to a week to detect changes in cell survival and growth as indicated by modulation of the luciferase signal. Effective AT₁ receptor antagonist compounds and doses and/or effective combinations with SERMs or other anti-estrogen compounds are identified by the difference in signal between the two cell lines in each individual mouse.

Methods of hollow fiber assay are described, for example, in the publications individually listed below and incorporated herein by reference.

Zhang & Kaelin (2005) Methods in Enzymology 399:530-549.

Zhang et al. (2004) Nature Medicine 10(6):643-648.

### Example 10

### ER Screening to Select Patients for AT₁ Receptor Antagonist Therapy

Tumor cells are obtained, for example by surgical biopsy, from a breast cancer patient and are screened by standard methods for estrogen receptors.

A patient having a tumor identified as ER+ is selected for therapy with an AT₁ receptor antagonist, either alone or in combination with one or more anti-estrogen agents and/or other anti-cancer agents that are known in the medical art.

A patient having only ER− tumors is selected not to receive AT₁ receptor antagonist therapy, but may receive therapy with other anti-cancer agents that are known in the medical art.

### Example 11

### ER Screening of SERM-Resistant Patients for at Receptor Antagonist Therapy

Tumor cells are obtained, for example by surgical biopsy, from a breast cancer patient who is or has been under SERM therapy but who has not achieved a completely satisfactory response (e.g., exhibiting tumor regression). The sample cells are screened by standard methods for estrogen receptors.

A patient having a tumor identified as ER+ is selected for therapy with an AT₁ receptor antagonist, either alone or in combination with one or more anti-estrogen agents other than SERMs (e.g., ER antagonists or aromatase inhibitors) and/or other anti-cancer agents that are known in the medical art.

A patient having only ER− tumors is selected not to receive AT₁ receptor antagonist therapy, but may receive therapy with other anti-cancer agents that are known in the medical art.

### Example 12

### Effect of AT₁ Receptor Antagonists on ER+ Breast Cancer Cell Line Xenografts

A study is conducted to evaluate effects of the AT₁ receptor antagonists candesartan cilexetil and irbesartan on growth properties of T47D xenografts in mice.

Female non-obese diabetic/severe combined immune-deficient (NODscid) mice of age 5-6 weeks are tumor-implanted by subcutaneous injection to the flank of ER+ human breast cancer cell line T47D (approximately 5×10⁶ cells/mouse). The mice are ear-notched for identification and housed 4 animals per cage. The tumor implantation site is palpated up to 3 times weekly to monitor tumor growth. Sixty animals in which tumor implantation has been successful (7-12 days after injection) are randomized to seven treatment groups:

1. vehicle (12 animals)

2. candesartan cilexetil 10 mg/kg (8 animals)

3. candesartan cilexetil 30 mg/kg (8 animals)

4. candesartan cilexetil 100 mg/kg (8 animals)

5. irbesartan 30 mg/kg (8 animals)

6. irbesartan 100 mg/kg (8 animals)

7. irbesartan 300 mg/kg (8 animals)

Dosing of AT₁ receptor antagonist or vehicle is p.o. (per os), once daily, for a duration of 3-5 weeks.

Tumors are measured 3 times weekly using digital calipers. Body weight is measured twice weekly. Clinical observations are conducted weekly. At the conclusion of the study, when mice attain a maximum tumor burden of about 1.5 cm³ or about 10% of body weight, the mice are sacrificed, tumors are harvested and a terminal blood sample is collected.

### Example 13

### Effect of AT₁ Receptor Antagonist, Aromatase Inhibitor and Combination of Both on Ang II-Induced Cell Proliferation

A cell proliferation assay was conducted by a procedure substantially as described in Example 2, using the ER+ human breast cancer cell line T47D. Following an eight-hour serum starvation phase, test substance was added to the cell culture to provide each of the following treatments:

vehicle control

Ang II, 500 DM

irbesartan (AT₁ receptor antagonist), 5 μM+Ang II, 500 nM

formestane (aromatase inhibitor), 10 μM+Ang II, 500 nM

formestane, 10 μM+irbesartan, 5 μM+Ang II, 500 nM

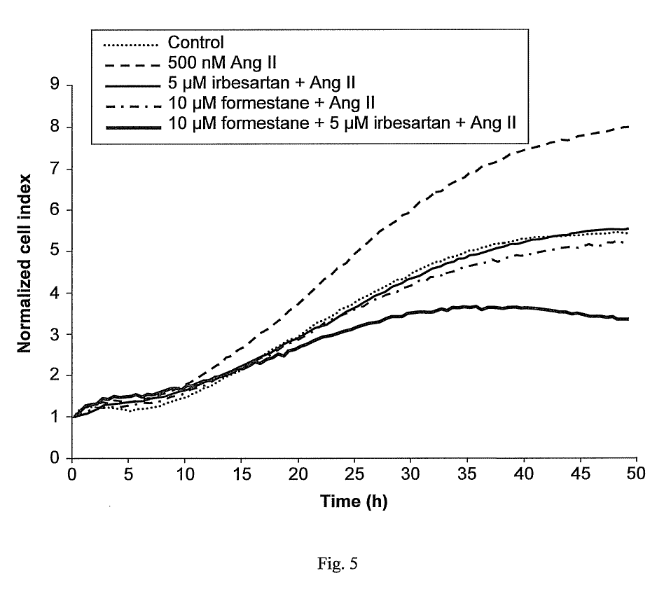

Results are shown in FIG. 5. Ang II induced a substantial increase in cell proliferation. Addition of either irbesartan or formestane reversed the Ang II effect, producing a result similar to that of vehicle control. Addition of a combination of formestane and irbesartan produced a substantially increased antiproliferative effect by comparison with either irbesartan or formestane alone.

### Example 14

### Effect of AT₁ Receptor Antagonist SERM and Combination of Both on Ang II-Induced Cell Proliferation

A cell proliferation assay was conducted by a procedure substantially as described in Example 2, using the ER+ human breast cancer cell line T47D. Following an eight-hour serum starvation phase, test substance was added to the cell culture to provide each of the following treatments:

vehicle control

Ang II, 500 nM

irbesartan (AT₁ receptor antagonist), 5 μM+Ang II, 500 nM

tamoxifen (SERM), 7.5 μM+Ang II, 500 nM

tamoxifen, 7.5 μM+irbesartan, 5 μM+Ang II, 500 nM

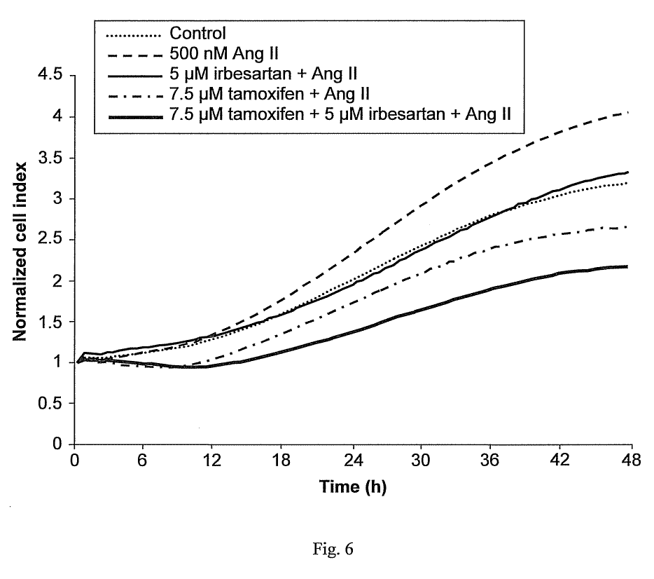

Results are shown in FIG. 6. Ang II induced a substantial increase in cell proliferation. Addition of irbesartan alone reversed the Ang II effect, producing a result similar to that of vehicle control. Addition of tamoxifen alone reduced cell proliferation to a level lower than that of vehicle control. Addition of a combination of tamoxifen and irbesartan produced an even greater antiproliferative effect than tamoxifen alone.

### Example 15

### Effect of AT₁ Receptor Antagonists on Growth of ER+ Breast Cancer Cell Line Xenografts

A study was conducted, similar in most respects to that described in Example 12 above, to evaluate effects of the AT₁ receptor antagonists candesartan cilexetil and irbesartan by comparison with the estrogen receptor antagonist tamoxifen (included as a positive control) on growth properties of T47D (human ER+ breast cancer cell line) xenografts in mice.

Female NODscid mice of age 5-6 weeks received a subcutaneous estrogen pellet implant (1.7 mg). Twenty four hours after estrogen pellet implantation, the mice received tumor implantation by subcutaneous injection of T47D cells to the flank (approximately 2.5×10⁶ cells/mouse). The mice were ear-notched for identification and housed 4 animals per cage. The tumor implantation site was palpated up to 3 times weekly to monitor tumor growth. One hundred animals in which tumor implantation was successful (7-12 days after injection) were randomized to ten treatment groups, 10 animals per group:

1. vehicle (50% peanut oil)

2. candesartan cilexetil (10 mg/kg)

3. candesartan cilexetil (30 mg/kg)

4. candesartan cilexetil (100 mg/kg)

5. irbesartan (30 mg/kg)

6. irbesartan (100 mg/kg)

7. irbesartan (300 mg/kg)

8. tamoxifen (0.1 mg/kg)

9. tamoxifen (0.3 mg/kg)

10. tamoxifen (1.0 mg/kg)

Dosing of AT₁ receptor antagonist, tamoxifen or vehicle was p.o., once daily, for a duration of 3-5 weeks. Tumors were measured 3 times weekly using digital calipers. Body weight was measured twice weekly. Clinical observations were conducted weekly. At the conclusion of the study or when mice attained a maximum tumor burden of about 3.0 cm³ or about 10% of body weight, the mice were sacrificed, tumors were harvested and a terminal blood sample was collected.

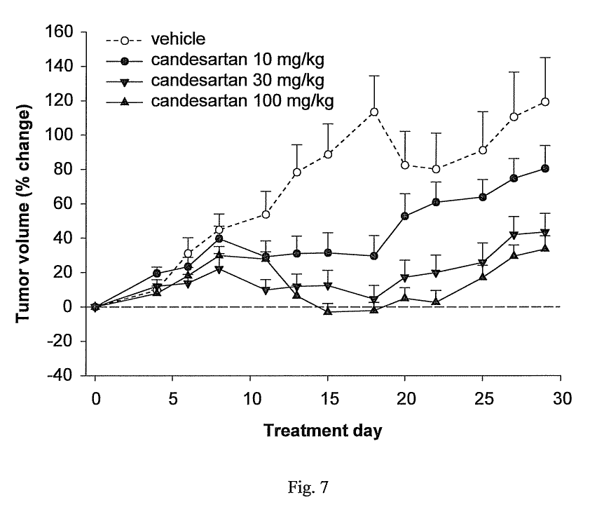

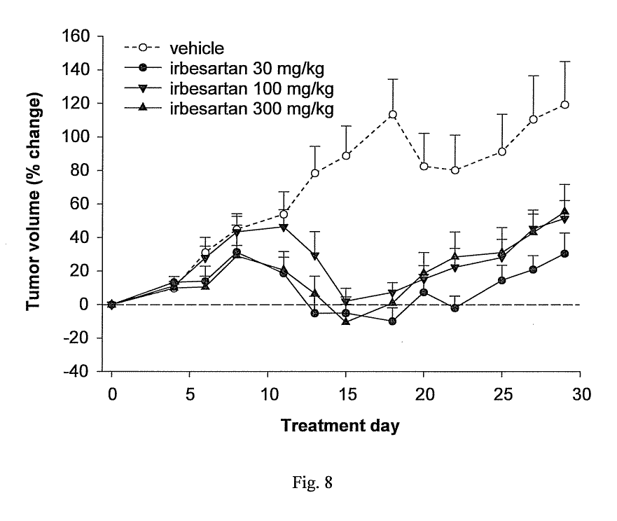

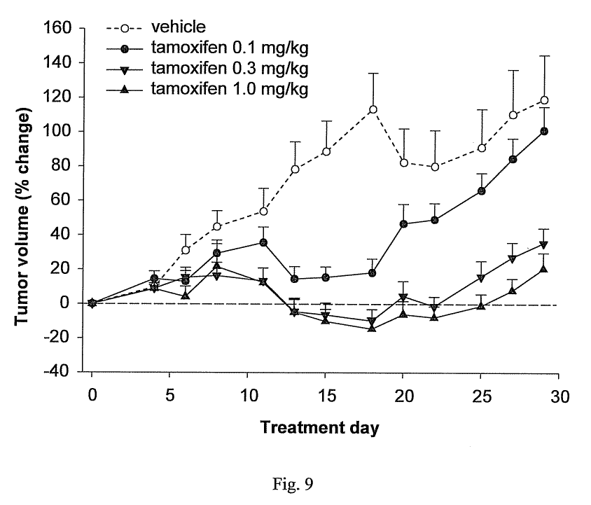

Results are shown in FIGS. 7 (candesartan cilexetil), 8 (irbesartan) and 9 (tamoxifen). Growth in tumor volume was reduced in a dose-dependent fashion by tamoxifen and by candesartan cilexetil, but in the case of irbesartan the lowest dose tested (30 mg/kg) appeared to give the greatest effect in this study.

### Example 16

### Effect of a Combination of AT₁ Receptor Antagonist and Tamoxifen on Growth of ER+ Breast Cancer Cell Line Xenografts

A study is conducted to evaluate effects of the AT₁ receptor antagonist candesartan cilexetil and the estrogen receptor antagonist tamoxifen, alone and in combination, on growth properties of T47D (human ER+ breast cancer cell line) xenografts in mice.

Female NODscid mice of age 5-6 weeks receive a subcutaneous estrogen pellet implant (1.7 mg). Twenty four hours after estrogen pellet implantation, the mice receive tumor implantation by subcutaneous injection of T47D cells to the flank (approximately 2.5×10⁶ cells/mouse). The mice are ear-notched for identification and housed 4 animals per cage. The tumor implantation site is palpated up to 3 times weekly to monitor tumor growth. Ninety animals in which tumor implantation has been successful (7-12 days after injection) are randomized to nine treatment groups, 10 animals per group:

1. vehicle (50% peanut oil)

2. tamoxifen (0.1 mg/kg)

3. tamoxifen (0.5 mg/kg)

4. candesartan cilexetil (10 mg/kg)

5. candesartan cilexetil (100 mg/kg)

6. tamoxifen (0.1 mg/kg)+candesartan cilexetil (10 mg/kg)

7. tamoxifen (0.1 mg/kg)+candesartan cilexetil (100 mg/kg)

8. tamoxifen (0.5 mg/kg)+candesartan cilexetil (10 mg/kg)

9. tamoxifen (0.5 mg/kg)+candesartan cilexetil (100 mg/kg)

All treatments are administered p.o., once daily, for a duration of 28 days following tumor formation. Tumors are measured 3 times weekly using digital calipers. Body weight is measured twice weekly. Clinical observations are conducted weekly. At the conclusion of the study or when mice attain a maximum tumor burden of about 3.0 cm³ or about 10% of body weight, the mice are sacrificed, tumors are harvested and a terminal blood sample is collected.

All patents and publications cited herein are incorporated by reference into this application in their entirety.

The words “comprise”, “comprises”, and “comprising” are to be interpreted inclusively rather than exclusively.

What is claimed is:

1. A method for treating breast cancer in a female patient, comprising
(a) determining whether the cancer comprises a tumor that is estrogen receptor and/or progesterone receptor positive;
(b) selecting the patient for AT₁ receptor antagonist therapy only if the cancer is determined to comprise an ER+ and/or PR+tumor; and
(c) administering to the patient, if so selected, an AT₁ receptor antagonist according to a regimen that is (i) effective to reduce growth, invasiveness and/or metastasis of the tumor and (ii) comprises a daily dose of the AT₁ receptor antagonist in the range of about 0.01 to about 100 mg/kg.

2. The method of claim 1, wherein the cancer is primary infiltrating ductal carcinoma.

3. The method of claim 1, wherein determination of presence of an ER+ and/or PR+tumor is made in a tissue sample of the patient by obtaining a positive result in an assay.

4. The method of claim 3, wherein the assay is selected from the group consisting of ligand binding assays, immunohistochemical assays and combinations thereof.

5. The method of claim 1, wherein the patient is selected for the AT₁ receptor antagonist therapy only if the cancer is determined to comprise an ER+ tumor.

6. The method of claim 1, further comprising determining, for a tumor found to be ER+, whether the tumor is resistant or responsive to selective estrogen receptor modulator (SERM) treatment.

7. The method of claim 1, wherein the AT₁ receptor antagonist administered comprises at least one compound selected from the group consisting of A-81282, A-81988, BMS-184,698, candesartan, CGP-49870, CI-996, CL-329,167, CP-161418, D-8731, DMP-581, DMP-811, DuP-532, E-4177, EMD-66397, eprosartan, EXP-408, EXP-970, EXP-3892, EXP-6803, EXP-7711, GA-0050, GR-138,950, HN-65,021, irbesartan, KRH-594, KT-3671, KW-3433, L-158,809, L-158,978, L-159,282, L-159,686, L-159,689, L-159,874, L-161,177, L-161,816, L-162,154, L-162,234, L-162,441, L-163,007, L-163,017, LF-7-0156, losartan, LR-B-081, LY-285,434, ME-3221, MK-996, olmesartan, PD-123,177, PD-123,319, PD-150,304, RWJ-38970, RWJ-46458, saprisartan, SC-48742, SC-50560, SC-51316, SC-51895, SC-52458, SL-910,102, TA-606, TAK-536, tasosartan, telmisartan, U-96,849, UP-269-6, UP-27,522, UR-7198, valsartan, WAY-126,227, WK-1492, XH-148, XR-510, YM-358, YM-31,472, ZD-8731, zolarsartan and pharmaceutically acceptable salts, prodrugs and active metabolites thereof.

8. The method of claim 1, wherein the administration regimen comprises a daily dose of the AT₁ receptor antagonist that is not greater than a normal maximum antihypertensive dose.

9. The method of claim 1, wherein the administration regimen comprises a daily dose of the AT₁ receptor antagonist that is greater than a normal maximum antihypertensive dose.

10. The method of claim 1, wherein the AT₁ receptor antagonist is administered in adjunctive or combination therapy with an estrogen receptor modulator or antagonist, antiprogestin and/or aromatase inhibitor.

11. The method of claim 10, wherein the AT₁ receptor antagonist is administered in adjunctive or combination therapy with a SERM comprising at least one compound selected from the group consisting of acolbifene, arzoxifene, bazedoxifene, droloxifene, HMR-3339, idoxifene, lasofoxifene, levormeloxifene, ospemifene, raloxifene, tamoxifen, toremifene, pharmaceutically acceptable salts, prodrugs and active metabolites thereof.

12. The method of claim 10, wherein the AT₁ receptor antagonist is administered in adjunctive or combination therapy with an aromatase inhibitor comprising at least one compound selected from the group consisting of aminoglutethimide, anastrozole, exemestane, fadrozole, formestane, letrozole, vorozole, pharmaceutically acceptable salts, prodrugs and active metabolites thereof.

13. The method of claim 1, wherein the AT₁ receptor antagonist is administered concomitantly with chemotherapy, radiotherapy and/or surgery to treat the cancer or a secondary tumor derived therefrom.

14. A therapeutic combination comprising an AT₁ receptor antagonist and a second agent that comprises an aromatase inhibitor or an estrogen receptor modulator or antagonist, in amounts effective in combination to reduce growth, invasiveness and/or metastasis of an ER+ breast tumor.

15. The combination of claim 14, wherein the AT₁ receptor antagonist comprises at least one compound selected from the group consisting of A-81282, A-81988, BMS-184,698, candesartan, CGP-49870, CI-996, CL-329,167, CP-161418, D-8731, DMP-581, DMP-811, DuP-532, E-4177, EMD-66397, eprosartan, EXP-408, EXP-970, EXP-3892, EXP-6803, EXP-7711, GA-0050, GR-138,950, HN-65,021, irbesartan, KRH-594, KT-3671, KW-3433, L-158,809, L-158,978, L-159,282, L-159,686, L-159,689, L-159,874, L-161,177, L-161,816, L-162,154, L-162,234, L-162,441, L-163,007, L-163,017, LF-7-0156, losartan, LR-B-081, LY-285,434, ME-3221, MK-996, olmesartan, PD-123,177, PD-123,319, PD-150,304, RWJ-38970, RWJ-46458, saprisartan, SC-48742, SC-50560, SC-51316, SC-51895, SC-52458, SL-910,102, TA-606, TAK-536, tasosartan, telmisartan, U-96,849, UP-269-6, UP-27,522, UR-7198, valsartan, WAY-126,227, WK-1492, XH-148, XR-510, YM-358, YM-31,472, ZD-8731, zolarsartan and pharmaceutically acceptable salts, prodrugs and active metabolites thereof.

16. The combination of claim 14, wherein the second agent comprises an aromatase inhibitor selected from the group consisting of aminoglutethimide, anastrozole, exemestane, fadrozole, formestane, letrozole, vorozole, pharmaceutically acceptable salts, prodrugs and active metabolites thereof.

17. The combination of claim 14, wherein the second agent comprises an estrogen receptor modulator or antagonist.

18. The combination of claim 17, wherein the estrogen receptor modulator or antagonist comprises a SERM selected from the group consisting of acolbifene, arzoxifene, bazedoxifene, droloxifene, HMR-3339, idoxifene, lasofoxifene, levormeloxifene, ospemifene, raloxifene, tamoxifen, toremifene, pharmaceutically acceptable salts, prodrugs and active metabolites thereof.

19. The combination of claim 17, wherein the estrogen receptor modulator or antagonist comprises fulvestrant or a pharmaceutically acceptable salts prodrug or active metabolite thereof.

20. The combination of claim 14, wherein the AT₁ receptor antagonist and the second agent are present in separate pharmaceutical compositions.

21. The combination of claim 14, wherein the AT₁ receptor antagonist and the second agent are present in a single pharmaceutical composition.

22. The combination of claim 21, wherein the composition further comprises at least one pharmaceutically acceptable excipient.
